SEQUENCE LISTING (9 sequences)

SEQ ID NO: 1 - protein, 6 aa, Rattus rattus
ESESRG

SEQ ID NO: 2 - protein, 6 aa, Rattus sp.
ESENVY

SEQ ID NO: 3 - protein, 883 aa, Rattus sp.
MIPLLLSLLAALVLTQAPAALADDLKEDSSEDRAFRVRIGAAQLRGVLGGALAIPCHVHHLRPPPSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACYGDMDGYPGVRNYGVVGPDDLYDVYCYAEDLNGELFLGAPPGKLTWEEARDYCLERGAQIASTGQLYAAWNGGLDRCSPGWLADGSVRYPIITPSQRCGGGLPGVKTLFLFPNQTGFPSKQNRFNVYCFRDSAHPSAFSEASSPASDGLEAIVTVTEKLEELQLPQEAVESENVYAIYSIPITEDGGGGSSTPEDPAEAPRTPLESETQSVAPPTGSSEEEGEALEEEERFKDTETPKEEKEQENLWVWPTELSSPLPTGLETEHSLSQVSPPAQAVLQLGASPSPRPPRVHGPPAETLQPPREGSLTSTPDGAREVAGETGSPELSGVPRESEEAGSSSLEDGPSLLPATWAPVGTRELETPSEEKSGRTVLTGTSVQAQPVLPTDSASRGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLAYAVDTVLRYRCRDGLAQRNLPLIRCQENGLWEAPQISCVPRRPARALRSMTAPEGPRGQLPRQRKALLTPPSSL

SEQ ID NO: 4 - DNA, 2652 bp, Rattus sp.
atgatcccattgcttctgtccctgctggcagctctggtcctgacccaagcccctgcagccctcgctgatgacctgaaagaagacagctcagaggatcgagcctttcgggtgcgcatcggtgccgcgcagctgcggggtgtgctgggcggtgccctggccatcccatgccacgtccaccacctgaggccgccgcccagccgccgggccgcgccgggctttccccgagtcaaatggaccttcctgtccggggaccgggaggtggaggtgctggtggcgcgcgggctgcgcgtcaaggtaaacgaagcctatcggttccgcgtggcgctgcctgcctaccccgcatcgctcacagatgtgtctttagtattgagcgaactgcggcccaatgattccggggtctatcgctgcgaggtccagcacggtatcgacgacagcagtgatgctgtggaagtcaaggtcaaaggggtcgtcttcctctaccgagagggctctgcccgctatgctttctccttcgctggagcccaggaagcctgtgctcgcatcggagcccgaattgccacccctgagcagctgtatgctgcctacctcggcggctatgaacagtgtgatgctggctggctgtccgaccaaaccgtgaggtaccccatccagaacccacgagaagcctgttatggagacatggatggctaccctggagtgcggaattacggagtggtgggtcctgatgatctctacgatgtctactgttatgccgaagacctaaatggagaactgttcctaggtgcccctcccggcaagctgacgtgggaggaggctcgggactactgtctggaacgcggtgctcagatcgctagcacgggccagctatacgcggcatggaatggcggcttggacagatgtagccctggctggctggctgatggcagtgtgcggtaccccatcatcacgcccagccaacgctgtgggggaggcctgccaggagtcaagaccctcttcctctttcccaaccagactggcttccccagcaagcagaaccgcttcaatgtctactgcttccgagactctgcccatccctctgccttctctgaggcctccagcccagcctctgatggactagaggccattgtcacagtgacagagaagctggaggaactgcagttgcctcaggaagctgtggagagcgagaatgtttacgcgatctactccatccccatcacagaagatgggggaggaggaagctctaccccagaagacccagcagaggcccccaggactcctctagaatcagaaacccaatccgttgcaccacctaccgggtcctcagaagaggaaggcgaagccctggaggaagaagaaagattcaaagacacagagactccgaaggaagagaaggagcaggagaacctgtgggtgtggcccacggagctcagcagccctctccctactggcttggaaacagagcactcactctcccaggtgtccccaccagcccaggcagttctacagctgggtgcatcaccttctcccaggcctccaagggtccatggaccgcctgcagagactttgcaacccccaagggagggaagcctcacatctactccagatggggcaagagaagtagcgggggaaactgggagccctgagctctctggggttcctcgagaaagcgaggaggcaggaagctccagcttggaggatggcccttccctccttccagcgacatgggcccctgtgggtaccagggagctggagaccccctcagaagagaagtctggaagaactgttctgacaggcacatcagtgcaggcccagccagtgctgcccaccgacagtgccagccgaggtggagtggctgtggctccctcatcaggtgactgtatccccagcccctgccacaatggtgggacatgcttggaggagaaggagggtttccgctgcctctgtttgccaggctatgggggggacctgtgcgatgttggcctccacttctgcagcccgggctgggaggccttccagggtgcctgctacaagcacttttccacacgaaggagttgggaggaggcagaaagccagtgccgagcgctaggggctcatctgaccagcatctgcacccctgaggagcaggactttgtcaacgatcgatacagggagtaccagtggattgggctcaatgacaggaccatcgagggtgacttcctgtggtcagatggtgcccctctgctctatgaaaactggaaccctgggcagcctgacagctacttcctgtctggggagaactgtgtggtcatggtgtggcatgaccagggacagtggagtgatgtaccctgcaactaccacctatcctacacctgcaagatggggcttgtgtcatgtggacctccaccacagctgcccctggctcaaatatttggtcgccctcggctggcctacgcggtggacactgtgcttcgatatcggtgccgagacgggctggcccagcgcaacttgccgttgatccgctgccaggagaatgggctttgggaggcccctcagatttcttgcgtgccccgaagacctgcccgtgctctccgctcaatgaccgccccagaaggaccacggggacagctcccgaggcagaggaaagcactgttgacacctccctccagtctctag

SEQ ID NO: 5 - DNA, 2652 bp, Rattus rattus
atgatcccattgcttctgtccctgctggcagctctggtcctgacccaagcccctgcagccctcgctgatgacctgaaagaagacagctcagaggatcgagcctttcgggtgcgcatcggtgccgcgcagctgcggggtgtgctgggcggtgccctggccatcccatgccacgtccaccacctgaggccgccgcccagccgccgggccgcgccgggctttccccgagtcaaatggaccttcctgtccggggaccgggaggtggaggtgctggtggcgcgcgggctgcgcgtcaaggtaaacgaagcctatcggttccgcgtggcgctgcctgcctaccccgcatcgctcacagatgtgtctttagtattgagcgaactgcggcccaatgattccggggtctatcgctgcgaggtccagcacggtatcgacgacagcagtgatgctgtggaagtcaaggtcaaaggggtcgtcttcctctaccgagagggctctgcccgctatgctttctccttcgctggagcccaggaagcctgtgctcgcatcggagcccgaattgccacccctgagcagctgtatgctgcctacctcggcggctatgaacagtgtgatgctggctggctgtccgaccaaaccgtgaggtaccccatccagaacccacgagaagcctgttatggagacatggatggctaccctggagtgcggaattacggagtggtgggtcctgatgatctctacgatgtctactgttatgccgaagacctaaatggagaactgttcctaggtgcccctcccggcaagctgacgtgggaggaggctcgggactactgtctggaacgcggtgctcagatcgctagcacgggccagctatacgcggcatggaatggcggcttggacagatgtagccctggctggctggctgatggcagtgtgcggtaccccatcatcacgcccagccaacgctgtgggggaggcctgccaggagtcaagaccctcttcctctttcccaaccagactggcttccccagcaagcagaaccgcttcaatgtctactgcttccgagactctgcccatccctctgccttctctgaggcctccagcccagcctctgatggactagaggccattgtcacagtgacagagaagctggaggaactgcagttgcctcaggaagctgtggagagcgagtctcgtggggcgatctactccatccccatcacagaagatgggggaggaggaagctctaccccagaagacccagcagaggcccccaggactcctctagaatcagaaacccaatccgttgcaccacctaccgggtcctcagaagaggaaggcgaagccctggaggaagaagaaagattcaaagacacagagactccgaaggaagagaaggagcaggagaacctgtgggtgtggcccacggagctcagcagccctctccctactggcttggaaacagagcactcactctcccaggtgtccccaccagcccaggcagttctacagctgggtgcatcaccttctcccaggcctccaagggtccatggaccgcctgcagagactttgcaacccccaagggagggaagcctcacatctactccagatggggcaagagaagtagcgggggaaactgggagccctgagctctctggggttcctcgagaaagcgaggaggcaggaagctccagcttggaggatggcccttccctccttccagcgacatgggcccctgtgggtaccagggagctggagaccccctcagaagagaagtctggaagaactgttctgacaggcacatcagtgcaggcccagccagtgctgcccaccgacagtgccagccgaggtggagtggctgtggctccctcatcaggtgactgtatccccagcccctgccacaatggtgggacatgcttggaggagaaggagggtttccgctgcctctgtttgccaggctatgggggggacctgtgcgatgttggcctccacttctgcagcccgggctgggaggccttccagggtgcctgctacaagcacttttccacacgaaggagttgggaggaggcagaaagccagtgccgagcgctaggggctcatctgaccagcatctgcacccctgaggagcaggactttgtcaacgatcgatacagggagtaccagtggattgggctcaatgacaggaccatcgagggtgacttcctgtggtcagatggtgcccctctgctctatgaaaactggaaccctgggcagcctgacagctacttcctgtctggggagaactgtgtggtcatggtgtggcatgaccagggacagtggagtgatgtaccctgcaactaccacctatcctacacctgcaagatggggcttgtgtcatgtggacctccaccacagctgcccctggctcaaatatttggtcgccctcggctggcctacgcggtggacactgtgcttcgatatcggtgccgagacgggctggcccagcgcaacttgccgttgatccgctgccaggagaatgggctttgggaggcccctcagatttcttgcgtgccccgaagacctgcccgtgctctccgctcaatgaccgccccagaaggaccacggggacagctcccgaggcagaggaaagcactgttgacacctccctccagtctctag

SEQ ID NO: 6 - protein, 883 aa, Rattus rattus
MIPLLLSLLAALVLTQAPAALADDLKEDSSEDRAFRVRIGAAQLRGVLGGALAIPCHVHHLRPPPSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACYGDMDGYPGVRNYGVVGPDDLYDVYCYAEDLNGELFLGAPPGKLTWEEARDYCLERGAQIASTGQLYAAWNGGLDRCSPGWLADGSVRYPIITPSQRCGGGLPGVKTLFLFPNQTGFPSKQNRFNVYCFRDSAHPSAFSEASSPASDGLEAIVTVTEKLEELQLPQEAVESESRGAIYSIPITEDGGGGSSTPEDPAEAPRTPLESETQSVAPPTGSSEEEGEALEEEERFKDTETPKEEKEQENLWVWPTELSSPLPTGLETEHSLSQVSPPAQAVLQLGASPSPRPPRVHGPPAETLQPPREGSLTSTPDGAREVAGETGSPELSGVPRESEEAGSSSLEDGPSLLPATWAPVGTRELETPSEEKSGRTVLTGTSVQAQPVLPTDSASRGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLAYAVDTVLRYRCRDGLAQRNLPLIRCQENGLWEAPQISCVPRRPARALRSMTAPEGPRGQLPRQRKALLTPPSSL

SEQ ID NO: 7 - DNA, 2878 bp, Homo sapiens
tgtggcactgcctgcgtacccaaccccagccctgggtagcctgcagcatggcccagctgttcctgcccctgctggcagccctggtcctggcccaggctcctgcagctttagcagatgttctggaaggagacagctcagaggaccgcgcttttcgcgtgcgcatcgcgggcgacgcgccactgcagggcgtgctcggcggcgccctcaccatcccttgccacgtccactacctgcggccaccgccgagccgccgggctgtgctgggctctccgcgggtcaagtggactttcctgtcccggggccgggaggcagaggtgctggtggcgcggggagtgcgcgtcaaggtgaacgaggcctaccggttccgcgtggcactgcctgcgtacccagcgtcgctcaccgacgtctccctggcgctgagcgagctgcgccccaacgactcaggtatctatcgctgtgaggtccagcacggcatcgatgacagcagcgacgctgtggaggtcaaggtcaaaggggtcgtctttctctaccgagagggctctgcccgctatgctttctccttttctggggcccaggaggcctgtgcccgcattggagcccacatcgccaccccggagcagctctatgccgcctaccttgggggctatgagcaatgtgatgctggctggctgtcggatcagaccgtgaggtatcccatccagaccccacgagaggcctgttacggagacatggatggcttccccggggtccggaactatggtgtggtggacccggatgacctctatgatgtgtactgttatgctgaagacctaaatggagaattgttcctgggtgaccctccagagaagctgacattggaggaagcacgggcgtactgccaggagcggggtgcagagattgccaccacgggccaactgtatgcagcctgggatggtggcctggaccactgcagcccagggtggctagctgatggcagtgtgcgctaccccatcgtcacacccagccagcgctgtggtgggggcttgcctggtgtcaagactctcttcctcttccccaaccagactggcttccccaataagcacagccgcttcaacgtctactgcttccgagactcggcccagccttctgccatccctgaggcctccaacccagcctccaacccagcctctgatggactagaggctatcgtcacagtgacagagaccctggaggaactgcagctgcctcaggaagccacagagagtgaatcccgtggggccatctactccatccccatcatggaggacggaggaggtggaagctccactccagaagacccagcagaggcccctaggacgctcctagaatttgaaacacaatccatggtaccgcccacggggttctcagaagaggaaggtaaggcattggaggaagaagagaaatatgaagatgaagaagagaaagaggaggaagaagaagaggaggaggtggaggatgaggctctgtgggcatggcccagcgagctcagcagcccgggccctgaggcctctctccccactgagccagcagcccaggaggagtcactctcccaggcgccagcaagggcagtcctgcagcctggtgcatcaccacttcctgatggagagtcagaagcttccaggcctccaagggtccatggaccacctactgagactctgcccactcccagggagaggaacctagcatccccatcaccttccactctggttgaggcaagagaggtgggggaggcaactggtggtcctgagctatctggggtccctcgaggagagagcgaggagacaggaagctccgagggtgccccttccctgcttccagccacacgggcccctgagggtaccagggagctggaggccccctctgaagataattctggaagaactgccccagcagggacctcagtgcaggcccagccagtgctgcccactgacagcgccagccgaggtggagtggccgtggtccccgcatcaggtgactgtgtccccagcccctgccacaatggtgggacatgcttggaggaggaggaaggggtccgctgcctatgtctgcctggctatgggggggacctgtgcgatgttggcctccgcttctgcaaccccggctgggacgccttccagggcgcctgctacaagcacttttccacacgaaggagctgggaggaggcagagacccagtgccggatgtacggcgcgcatctggccagcatcagcacacccgaggaacaggacttcatcaacaaccggtaccgggagtaccagtggatcggactcaacgacaggaccatcgaaggcgacttcttgtggtcggatggcgtccccctgctctatgagaactggaaccctgggcagcctgacagctacttcctgtctggagagaactgcgtggtcatggtgtggcatgatcagggacaatggagtgacgtgccctgcaactaccacctgtcctacacctgcaagatggggctggtgtcctgtgggccgccaccggagctgcccctggctcaagtgttcggccgcccacggctgcgctatgaggtggacactgtgcttcgctaccggtgccgggaaggactggcccagcgcaatctgccgctgatccgatgccaagagaacggtcgttgggaggccccccagatctcctgtgtgcccagaagacctgcccgagctctgcacccagaggaggacccagaaggacgtcaggggaggctactgggacgctggaaggcgctgttgatccccccttccagccccatgccaggtccctagggggcaaggccttgaacactgccggccacagcactgccctgtcacccaaattttccctcacaccctgcgctcaccacaggaagtgacaacatgac

SEQ ID NO: 8 - protein, 911 aa, Homo sapiens
MAQLFLPLLAALVLAQAPAALADVLEGDSSEDRAFRVRIAGDAPLQGVLGGALTIPCHVHYLRPPPSRRAVLGSPRVKWTFLSRGREAEVLVARGVRVKVNEAYRFRVALPAYPASLTDVSLALSELRPNDSGIYRCEVQHGIDDSSDAVEVKVKGVVFLYREGSARYAFSFSGAQEACARIGAHIATPEQLYAAYLGGYEQCDAGWLSDQTVRYPIQTPREACYGDMDGFPGVRNYGVVDPDDLYDVYCYAEDLNGELFLGDPPEKLTLEEARAYCQERGAEIATTGQLYAAWDGGLDHCSPGWLADGSVRYPIVTPSQRCGGGLPGVKTLFLFPNQTGFPNKHSRFNVYCFRDSAQPSAIPEASNPASNPASDGLEAIVTVTETLEELQLPQEATESESRGAIYSIPIMEDGGGGSSTPEDPAEAPRTLLEFETQSMVPPTGFSEEEGKALEEEEKYEDEEEKEEEEEEEEVEDEALWAWPSELSSPGPEASLPTEPAAQEESLSQAPARAVLQPGASPLPDGESEASRPPRVHGPPTETLPTPRERNLASPSPSTLVEAREVGEATGGPELSGVPRGESEETGSSEGAPSLLPATRAPEGTRELEAPSEDNSGRTAPAGTSVQAQPVLPTDSASRGGVAVVPASGDCVPSPCHNGGTCLEEEEGVRCLCLPGYGGDLCDVGLRFCNPGWDAFQGACYKHFSTRRSWEEAETQCRMYGAHLASISTPEEQDFINNRYREYQWIGLNDRTIEGDFLWSDGVPLLYENWNPGQPDSYFLSGENCVVMVWHDQGQWSDVPCNYHLSYTCKMGLVSCGPPPELPLAQVFGRPRLRYEVDTVLRYRCREGLAQRNLPLIRCQENGRWEAPQISCVPRRPARALHPEEDPEGRQGRLLGRWKALLIPPSSPMPGP

SEQ ID NO: 9 - protein, 7 aa, Rattus sp.
QEAVESE

(19) United States
(12) Patent Application Publication
(10) Pub. No.: US 2009/0117595 A1
(43) Pub. Date: May 7, 2009

(54) PRIMARY CENTRAL NERVOUS SYSTEM TUMOR SPECIFIC BEHAB ISOFORMS

(75) Inventors: Susan Hockfield, Cambridge, MA (US); Russell T. Matthews, Skaneateles, NY (US); Mariano S. Viapiano, New Haven, OH (US)

Correspondence Address: ROPES & GRAY LLP, PATENT DOCKETING 39/361, 1211 AVENUE OF THE AMERICAS, NEW YORK, NY, 10036-8704, US

(73) Assignee: Yale University, New Haven, CT (US)

(21) Appl. No.: 10/586,196

(22) PCT Filed: Jan. 14, 2005
(86) PCT No.: PCT/US2005/001184

§ 371 Date: Dec. 5, 2008

Related U.S. Application Data
(60) Provisional application No. 60/536,594, filed on Jan. 15, 2004.

(51) Int. Cl. G01N 33/574; C07K 14/00; G01N 33/566

(52) U.S. Cl. 435/7.23; 530/395; 436/501

(57) ABSTRACT

The present invention comprises compositions and methods related to a glycosylation-variant BEHAB protein, poly-sialyated full-length BEHAB, and methods of use thereof.

### STATEMENT REGARDING FEDERALLY SPONSORED RESEARCH OR DEVELOPMENT

This invention was supported in part by funds obtained from the U.S. Government (National Institutes of Health Grant Number RO1 NS35228) and the U.S. Government may therefore have certain rights in the invention.

### BACKGROUND OF THE INVENTION

Gliomas are glial tumors derived from astrocytic, oligodendroglial and ependymal cells. Gliomas are notoriously deadly brain tumors characterized by their diffuse invasion into the surrounding normal brain tissue. Further, gliomas constitute the most common form of primary CNS tumors and include several histologically distinct subtypes, most of them malignant and highly invasive (Kleihues et al., 2002, J. Neuropathol Exp Neurol, 61: 215-229). The most dangerous property of malignant gliomas is their highly invasive phenotype, which makes these primary brain tumors difficult to control and impossible to completely remove by surgery, thus accounting for the high lethality of gliomas (Pilkington, 1996, Braz J Med Biol Res, 29: 1159-72; Giese and Westphal, 1996, Neurosurgery 39: 235-252). Glioblastomas, the most common and most aggressive class of gliomas, result in patient's death typically within one year of diagnosis, due to the inevitable recurrence even after extensive resection (Bernstein and Woodard, 1995, Neurosurgery, 36: 124-132, 1995). Despite considerable advances in the understanding of these tumors, the survival rates for patients with gliomas have remained essentially unchanged for 25 years (Berens and Giese, 1999, Neoplasia, 1: 208-19). Novel therapeutic strategies will follow from an understanding of the mechanisms and molecules involved in glioma invasion.

The invasive behavior of glioma cells in the central nervous system (CNS) is quite unusual, in that the adult CNS is highly restrictive to cell movement even for non-glial tumors that metastasize to the brain (Pilkington, 1997, Anticancer Res. 17: 4103-4105; Subramanian et al., 2002, Lancet Oncol. 3: 498-507). The unique ability of gliomas to infiltrate and invade the surrounding normal neural tissue indicates that these cells are able to overcome the normal barriers to cell movement in the CNS (Giese and Westphal, 1996, Neurosurgery 39: 235-252).

One of the major barriers to cell movement in all tissues, including the CNS, is the extracellular matrix (ECM). The ECM of the CNS is composed of a hyaluronic acid (HA) scaffold associated to glycoproteins and proteoglycans (Celio and Blumcke, 1994, Brain Res Brain Res Rev. 19: 128-45). Classical fibrous ECM proteins such as laminin, type IV collagen, fibronectin and vitronectin are limited to vascular basal membranes and the glia limitans in the adult CNS and are essentially absent from the parenquima (Gladson, 1999, J. Neuropathol Exp Neurol, 58: 1029-40). Interaction of glioma cells with this HA-based ECM is mediated by several cell surface receptors such as CD44, RHAMM and proteoglycans members of the lectican family (Goldbrunner et al., 1999, Acta Neurochir (Wien) 141: 295-305; Novak and Kaye, 2000, J. Clin Neurosci, 7: 280-90; Akiyama et al., 2001, J. Neurooncol. 53: 115-27) including BEHAB/brevican (BEHAB) (Yamaguchi, 2000, Cell Mol Life Sci. 57: 276-89).

BEHAB is a CNS-specific extracellular chondroitin sulfate proteoglycan that is expressed in a spatially- and temporally-regulated manner in the mammalian brain (Jaworski et al., 1994, J. Cell Biol. 125: 495-509). BEHAB expression is upregulated in the ventricular zone coincident with gliogenesis (Jaworski et al., 1995, J. Neurosci. 15: 1352-1362), and during reactive gliosis after a stab injury (Jaworski et al., 1999, Exp Neurol. 157: 327-37), indicating that this proteoglycan is involved in glial cell proliferation and/or motility. Consistent with these findings, BEHAB mRNA expression is also dramatically upregulated in surgical samples of human glioma as well as in a rodent glioma model (Jaworski et al., 1996, Cancer Res. 56: 2293-2298). Further, BEHAB upregulation and its subsequent proteolytic processing contribute to the invasive phenotype of glioma (Zhang et al., 1998, J. Neurosci. 18: 2370-2376; Nutt et al., 2001, Neuroscientist, 7: 113-122). However, a further understanding of the molecular interactions and mechanisms through which the functions of BEHAB are mediated is still required.

One of the difficulties in characterizing the functions of BEHAB is the molecular complexity of this protein. Different isoforms of BEHAB have been described, resulting from alternative splicing (Seidenbecher et al., 1995, J. Biol. Chem. 270: 27206-27212), proteolytic cleavage (Nakamura et al., 2000, J. Biol. Chem., 275: 38885-38890; Matthews et al., 2000, J. Biol. Chem. 275: 22695-22703), and/or differential glycosylation of the core protein (Yamada et al., 1994, J. Biol. Chem. 269: 10119-10126; Viapiano et al., 2003, J. Biol. Chem., 278: 33239-33247). It is likely that these isoforms may interact differently with the cell surface and with other ECM components, and thus play unique roles in glioma progression.

A novel glycoform of BEHAB/brevican, named rat glycosylation-variant BEHAB (B/b₁₃₀), which is underglycosylated and highly expressed early in development, was discovered in the rat brain (Id.). Importantly, this isoform is the major BEHAB isoform upregulated in a rat experimental model of invasive glioma.

Almost all cancers are characterized by aberrant glycosylation of cell surface proteins (Hakomori, 2002, Proc. Natl Acad Sci USA 99: 10231-10233). Changes in glycosylation disrupt the normal protein-protein interactions and therefore can be associated to tumor invasion and metastasis (Kim and Varki, 1997, Glycoconj. J. 14: 569-576; Gorelik et al., 2001, Cancer Metastasis Rev. 20: 245-277).

Aberrant glycosylation is identified by the appearance of either truncated versions of normal oligosaccharides or unusual types of terminal oligosaccharide sequences (e.g., Lewis^{x/a}). These changes may equally affect N- and O-linked oligosaccharides (Burchell et al., 2001, J. Mammary Gland Biol Neoplasia 6: 355-364 2001; Dwek et al., 2001, Proteomics 1: 756-62). In particular, a general increase in the appearance of alpha 2,6- and alpha 2,3-linked sialic acid is a common feature of tumors (Narayanan, 1994, Ann Clin Lab Sci. 24: 376-384), including glioma (Reboul et al., 1996, Glycoconj. J. 13: 69-79; Yamamoto et al., 1997, Brain Res., 755: 175-179), and has been associated to an increase in the metastatic ability of certain cancers.

Lack of specific oligosaccharides in tumors, though less commonly noted, has also been described (see Dennis, 1986, Cancer Res. 46: 4594-4600; Dabelsteen et al., 1991, J. Oral Pathol Med. 20: 361-368; Ciborowski and Finn, 2002, Clin. Exp Metastasis 19: 339-45). An interesting example is represented by the cell-surface receptors with aberrantly underglycosylated neo-glycoforms in CNS tumors that cannot bind their normal ligands (e.g., CD44H in neuroblastoma (Gross et al., 2001, Med. Pediatr Oncol. 36: 139-41).

Targeting tumor cells selectively through their specific cell-surface antigens is an approach that is regaining popularity as a cancer therapy. Considerable research over the past decade has made great progress in demonstrating the utility of antibody immunotherapy in the treatment of many tumor types (see Carter, 2001, Nat. Rev Cancer, 1: 118-129), including glioma (Kurpad et al., 1995, Glia 15: 244-256; Kuan et al., 2001, Endocr. Relat Cancer, 8: 83-96; Goetz et al., 2003, J. Neurooncol. 62: 321-328). Given the refractory properties of gliomas to traditional chemo- and radiotherapy, immunotherapy is a promising treatment for primary CNS tumors. However, a hurdle in using this approach as a therapy for glioma has been the lack of good cellular targets that are both restricted to the tumor cells and available at the cell surface for targeting (Yang et al., 2003, Cancer Control. 10: 138-147). Among those that have been proposed (Kurpad et al., 1995, Glia 15: 244-256), and clinically explored are the deletion mutant EGF receptor (EGFRvIII, reviewed in Kuan et al., (2001, Endocr. Relat Cancer, 8: 83-96)), which is expressed in ˜50% of all glioblastomas (Kurpad et al., 1995, Glia 15: 244-256), and the extracellular matrix protein tenascin-C, which is highly upregulated in >90% of all gliomas compared to normal brain (McLendon et al., 2000, J. Histochem Cytochem. 48: 1103-1110).

Given the high mortality associated with primary CNS tumors, such as glioma, and the paucity of effective therapies, there exists a long felt need for molecular targets on primary CNS tumors to aid in the diagnosis and treatment of these neoplasias. The present invention meets this need.

### BRIEF SUMMARY OF THE INVENTION

The present invention comprises an isolated human poly-sialyated full-length BEHAB isoform, wherein the poly-sialyated full-length BEHAB isoform has a molecular weight greater than about 160 kDa.

In one aspect of the present invention, the nucleic acid encoding the poly-sialyated full-length BEHAB isoform comprises the isolated nucleic acid of SEQ ID NO:7.

The present invention further comprises a method of detecting a primary CNS tumor in a mammal, the method comprising contacting a biological sample of the mammal with an antibody that specifically binds with a poly-sialyated full-length BEHAB isoform, or fragment thereof, and detecting binding of the antibody to the biological sample, wherein binding of the antibody with the biological sample detects a primary CNS tumor in a mammal.

In one aspect, the mammal is a human.

In another aspect, the biological sample is a CNS tissue sample.

In yet another aspect, the CNS tissue sample is a brain tissue.

In still another aspect, the antibody is selected from the group consisting of B5, B6, and B_{CRP}.

In one aspect, the antibody comprises a tag polypeptide covalently linked thereto.

In another aspect, the primary CNS tumor is a glioma.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A and FIG. 1B, is a series of images depicting immunoblots demonstrating the identification of a novel, membrane-associated human glycosylation-variant BEHAB isoform in human glioma. FIG. 1A depicts a total homogenate (H) from a postmortem sample from a normal, age-matched human brain (Control), FIG. 1B depicts a total homogenate (H) from a surgical sample of a representative glioblastoma multiforme (Glioma). The membrane-enriched pellet and final soluble fraction are indicated by M and S, respectively. Arrows indicate the positions of a full-length BEHAB (B/b), a poly-sialylated full-length BEHAB (B/bₛᵢₐ) and a membrane-associated glycosylation-variant BEHAB isoform (B/b_{Δg}) only observed in the glioma sample.

FIG. 2, comprising FIG. 2A and FIG. 2B, is a series of images depicting immunoblots indicating that glycosylation-variant BEHAB (B/b_{Δg}) is absent from normal human brain throughout development. FIG. 2A depicts total homogenates from postmortem samples of human brain cortex over various developmental periods (1 to 76 years old). FIG. 2B depicts total homogenates from human brain cortex over prenatal and early postnatal development (16 gestational weeks to 1 year old), and a representative surgical sample from a glioblastoma multiforme (Glioma).

FIG. 3, comprising FIG. 3A, FIG. 3B and FIG. 3C is a series of images depicting that glycosylation-variant BEHAB (B/b_{Δg}) is a membrane-associated, glioma-specific isoform of BEHAB. FIG. 3A is an image depicting a representative subset (out of total n=21) surgical samples of high-grade human glioma. Arrows indicate the positions of the 160-kDa full-length BEHAB (B/b) isoform as well as the membrane-associated isoform glycosylation variant BEHAB. Asterisks indicate the position of poly-sialyated full-length BEHAB (B/bₛᵢₐ). FIG. 3B is an image depicting total homogenates from samples of several non-glial neuropathologies, indicating the presence of full-length BEHAB in the epilepsy foci (epilepsy) and Alzheimer's disease (AD) samples, but the absence of glycosylation-variant BEHAB in any of the samples analyzed. BEHAB expression is absent in samples from epidermoid tumor (epid), meningioma (meng), schwannoma (acoustic neuroma, neur) and medulloblastoma (medul). FIG. 3C is a graph depicting optical densitometry of glioma samples (age 43-52 years, n=8) and age-matched controls (age 46-51 years, n=5) for full-length BEHAB (B/b) and glycosylation-variant BEHAB (B/b_{Δg}). Total B/b=full-length B/b+B/bDg. Integrated optical density (IOD) of B/b and B/bₛᵢₐ were added to account for full-length B/b in gliomas.

FIG. 4, comprising FIG. 4A, FIG. 4B and FIG. 4C, is a series of images depicting that glycosylation-variant BEHAB is a full-length isoform of BEHAB. FIG. 4A is a schematic diagram depicting the structure of full-length BEHAB and the location of the epitopes recognized by the antibodies B6, B5 and BCRP, the location of the HA-binding domain (HABD), the chondroitin sulfate attachment region (GAG), the epidermal growth factor repeat (EGF), and the complement regulatory protein-like domain (CRP). FIG. 4B is an image of an immunoblot depicting solubilized brain membranes (M) from control and glioma samples immunoprecipitated in the absence (mock) or presence (B6) of B6 antibody. FIG. 4C is an image of an immunoblot depicting culture medium (m) and cell membranes (c) from the human glioma cell line U87-MG, transiently transfected with a full-length human BEHAB cDNA, and immunoblotted with B6, B5 and BCRP antibodies.

FIG. 5, comprising FIGS. 5A and 5B, is a series of images demonstrating that human glycosylation-variant BEHAB (B/b_{Δg}) is produced by differential glycosylation of a BEHAB core protein. FIG. 5A depicts soluble and particulate fractions from a control and FIG. 5B depicts soluble and particulate fractions from glioma samples. Samples were treated with chondroitinase ABC alone (CH'ase) or with the addition of PNGase F (PNG-F), O-glycosidase (O-glycos), sialidase or all the enzymes combined. Arrows indicate full-length BEHAB (B/b) and human glycosylation variant BEHAB (B/b_{Δg}).

FIG. 6, comprising FIGS. 6A and 6B, is a series of images depicting the association of the peripheral human glycosylation-variant BEHAB (B/b_{Δg}) with the cell membranes in a calcium-independent manner. Total membranes (M) from control and glioma samples were centrifuged and the resulting supernatant (s) and pellet (p) were processed for Western blotting.

FIG. 7, comprising FIGS. 7A through 7E, is a series of images depicting the location of human glycosylation-variant BEHAB on the cell surface. Untreated U87-MG cells transfected with full-length human BEHAB cDNA were live-stained and further processed for immunocytochemistry and fluorescence detection. Cells were probed with an anti-BEHAB antibody B6 (B6), counterstained with DAPI (DAPI), and the images were merged (merge). FIG. 7A and FIG. 7B depict cells transfected with human full-length BEHAB cDNA and stained with an anti-BEHAB antibody, FIG. 7C depicts control (vector)-transfected cells stained with an anti-BEHAB antibody and FIG. 7D depicts cells transfected with human full-length BEHAB cDNA and stained with non-immune serum. The bar=25 μm. FIG. 7E is an image of an immunoblot of total homogenates (homog.) from human full-length BEHAB cDNA-transfected (B/b) versus control (Ctrl) cells depicting the presence of only human glycosylation-variant BEHAB in cells processed for live staining when compared to culture medium (m) and cell membranes (c) from separate cells transfected with human full-length BEHAB cDNA.

FIG. 8 is an image depicting SEQ ID NO:1, a peptide.

FIG. 9 is an image depicting SEQ ID NO:2, a peptide.

FIG. 10 is an image depicting SEQ ID NO:3, a mammalian mutant BEHAB polypeptide.

FIG. 11 is an image depicting SEQ ID NO:4, a mammalian mutant BEHAB nucleic acid.

FIG. 12 is an image depicting SEQ ID NO:5, a rat BEHAB nucleic acid.

FIG. 13 is an image depicting SEQ ID NO:6, a rat BEHAB polypeptide.

FIG. 14 is an image depicting SEQ ID NO:7, a human BEHAB nucleic acid.

FIG. 15 is an image depicting SEQ ID NO:8, a human BEHAB polypeptide.

### DETAILED DESCRIPTION OF THE INVENTION

The present invention is based on the discovery of a novel underglycosylated or unglycosylated BEHAB isoform in human brain termed glycosylation-variant BEHAB, which, as demonstrated by the data disclosed herein, is absent from the normal adult brain and neuropathologic control and is highly over-expressed in surgical samples from human glioblastoma. As disclosed herein, the novel glycosylation-variant BEHAB isoform in human brain is ˜10-kDa smaller than full-length BEHAB but it is not a cleavage product or splice variant of the full-length protein. Instead, as evidenced by the data disclosed elsewhere herein, human glycosylation-variant BEHAB is an under- or unglycosylated isoform that lacks most, if not all, of the carbohydrates with which it is typically invested. Despite the lack of glycosylation, human glycosylation variant BEHAB is found on the extracellular surface of cells and binds via a mechanism unique from other known BEHAB isoforms. As disclosed elsewhere herein, glycosylation-variant BEHAB can play a unique role in glioma progression and can be a relevant cell-surface target for immuno-therapy.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Amplification” refers to any means by which a polynucleotide sequence is copied and thus expanded into a larger number of polynucleotide molecules, e.g., by reverse transcription, polymerase chain reaction, and ligase chain reaction.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator” as the term is used herein, is meant any device including, but not limited to, a hypodermic syringe, a pipette, and the like, for administering the mutant BEHAB nucleic acid, protein, and/or anti-BEHAB antibodies and the antisense BEHAB nucleic acid of the invention to a mammal.

“BEHAB” “full-length BEHAB”, or “endogenous BEHAB” as the terms are used synonymously herein, refers to the Brain-Enriched Hyaluronan Binding molecule, otherwise known as brevican. Full-length BEHAB has a molecular weight of greater than about 150 kDa in rats and mice, and greater than about 160 kDa in humans and is exemplified by the nucleotide and amino acid sequences set forth in SEQ ID NO:5 SEQ ID NO:6 for rat full-length BEHAB, and SEQ ID NO:7 and SEQ ID NO:8 for human full-length BEHAB, respectively.

“Biological sample,” as that term is used herein, means a sample obtained from or in a mammal that can be used to assess the level of expression of a BEHAB, the level of BEHAB protein present, or both. Such a sample includes, but is not limited to, a blood sample, a neural tissue sample, a brain sample, and a cerebrospinal fluid sample.

“Cleavage” is used herein to refer to the disassociation of a peptide bond between two amino acids in a polypeptide, thereby separating the polypeptide comprising the two amino acids into at least two fragments.

A “cleavage inhibitor” is used herein to refer to a molecule, compound or composition that prevents the cleavage of a polypeptide either by titrating the protease responsible for cleavage, blocking the cleavage site, or otherwise making the cleavage site unrecognizable to a protease.

“Cleavage inhibiting amount” is used herein to refer to an effective amount of a cleavage inhibitor.

“Cleavage products” is used herein to refer to the fragments of an initial polypeptide resulting from the cleavage of the initial polypeptide into two or more fragments. As an example, the cleavage products of the 145 kDa BEHAB protein include 90 kDa and 50 kDa fragments.

By “complementary to a portion or all of the nucleic acid encoding BEHAB” is meant a sequence of nucleic acid which does not encode a BEHAB protein. Rather, the sequence which is being expressed in the cells is identical to the non-coding strand of the nucleic acid encoding a BEHAB protein and thus, does not encode BEHAB protein.

The terms “complementary” and “antisense” as used herein, are not entirely synonymous. “Antisense” refers particularly to the nucleic acid sequence of the non-coding strand of a double stranded DNA molecule encoding a protein, or to a sequence which is substantially homologous to the non-coding strand.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g. amino acid residues in a protein export signal sequence).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A first region of an oligonucleotide “flanks”—a second region of the oligonucleotide if the two regions are adjacent one another or if the two regions are separated by no more than about 1000 nucleotide residues, and preferably no more than about 100 nucleotide residues.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 500 nucleotides, even more preferably, at least about 500 nucleotides to about 1000 nucleotides, yet even more preferably, at least about 1000 to about 1500, even more preferably, at least about 1500 nucleotides to about 2000 nucleotides, yet even more preferably, at least about 2000 to about 2500, even more preferably, at least about 2500 nucleotides to about 2600 nucleotides, yet even more preferably, at least about 2600 to about 2650, and most preferably, the nucleic acid fragment will be greater than about 2652 nucleotides in length.

As applied to a protein, a “fragment” of BEHAB is about 20 amino acids in length. More preferably, the fragment of a BEHAB is about 100 amino acids, even more preferably, at least about 200, yet more preferably, at least about 300, even more preferably, at least about 400, yet more preferably, at least about 500, even more preferably, about 600, and more preferably, even more preferably, at least about 700, yet more preferably, at least about 800, even more preferably, about 850, and more preferably, at least about 884 amino acids in length.

As used herein, a “glycosylation-variant BEHAB isoform” and “glycosylation-variant BEHAB” means a BEHAB protein having an altered glycosylation pattern as compared to the glycosylation pattern of full-length BEHAB and a molecular weight less than about 150 kDa in rats and less than about 160 kDa in humans. The term glycosylation-variant BEHAB isoform or glycosylation-variant BEHAB includes underglycosylated BEHAB, differently-glycosylated BEHAB and unglycosylated BEHAB.

As used herein, a “poly-sialyated full-length BEHAB” and “poly-sialyated full-length BEHAB isoform” means a BEHAB protein having an altered glycosylation pattern as compared to the glycosylation pattern of full-length BEHAB and a molecular weight greater than about 160 kDa in humans when not treated with sialidase.

As used herein, a “differently-glycosylated BEHAB” and a “differently-glycosylated BEHAB isoform” refers to a BEHAB protein having an altered glycosylation pattern wherein the carbohydrate and sugar content is similar to that of full-length BEHAB, but the composition of the sugars associated with the amino acid backbone is altered.

“Underglycosylated BEHAB isoform” and “underglycosylated BEHAB” are used herein to refer to a BEHAB protein having the primary amino acid sequence of a full-length BEHAB protein, or a fragment thereof, but having less than the glycosylation content of the full-length BEHAB protein, but still having at least one sugar or carbohydrate associated with the protein.

“Unglycosylated BEHAB isoform” and “unglycosylated BEHAB” are used herein to refer to a BEHAB protein having the primary amino acid sequence of a full-length BEHAB protein, or fragment thereof, but having no sugars or carbohydrates associated with the protein.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

“Mutant BEHAB” is used herein to refer to a Brain Enriched Hyaluronan Binding molecule in which the amino acid sequence has been modified to inhibit cleavage by proteases.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is naturally-occurring.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least at least about twenty sequential nucleotide residues of the polynucleotide. It is understood that a portion of a polynucleotide may include every nucleotide residue of the polynucleotide.

“Primary CNS tumor” is used herein to refer to a neoplasia with origins in the brain, in that the cancerous cells did not originate in another part of the body and metastasize to the brain. Examples of primary CNS tumors include, but are not limited to, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, reactive gliosis, primitive neuroectodermal tumors, schwannomas, lymphomas, vascular tumors, and lymphomas.

“Treating a primary CNS tumor” is used herein to refer to a situation where the severity of a symptom of a primary CNS tumor, including the volume of the tumor or the frequency with which any symptom or sign of the tumor is experienced by a patient, or both, is reduced, or where time to tumor progression or survival time is increased.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

By the term “specifically binds,” as used herein, is meant an antibody which recognizes and binds an epitope of a BEHAB protein, but does not substantially recognize or bind other molecules in a sample.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

A “transgene”, as used herein, means an exogenous nucleic acid sequence comprising a nucleic acid which encodes a promoter/regulatory sequence operably linked to nucleic acid which encodes an amino acid sequence, which exogenous nucleic acid is encoded by an animal or cell.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Description

### 1. Isolated Nucleic Acids

A. Sense Nucleic Acids

The present invention includes an isolated nucleic acid encoding a glycosylation-variant BEHAB molecule, or a fragment thereof, wherein the nucleic acid shares some identity with a nucleic acid having the sequence of SEQ ID NO:7. Preferably, the nucleic acid is about 1% homologous, more preferably, about 5% homologous, even more preferably about 10% homologous, even more preferably about 20% homologous, even more preferably, the nucleic acid is about 30% homologous, more preferably, about 40% homologous, even more preferably about 50% homologous, even more preferably, the nucleic acid is about 60% homologous, more preferably, about 70% homologous, even more preferably about 80% homologous. Preferably, the nucleic acid is about 90% homologous, more preferably, about 95% homologous, even more preferably about 99% homologous, even more preferably about 99.9% homologous to SEQ ID NO:7, disclosed herein. Even more preferably, the nucleic acid is SEQ ID NO:7. The isolated nucleic acid of the invention should be construed to include an RNA or a DNA sequence encoding a glycosylation-variant BEHAB protein of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The present invention should not be construed as being limited solely to the nucleic and amino acid sequences disclosed herein. Once armed with the present invention, it is readily apparent to one skilled in the art that other nucleic acids encoding a glycosylation-variant BEHAB protein, including a polysialyated full-length BEHAB protein can be obtained by following the procedures described herein in the experimental details section for the generation of other glycosylation-variant BEHAB nucleic acids encoding glycosylation-variant BEHAB polypeptides as disclosed herein and procedures that are well-known in the art or to be developed.

Further, any other number of procedures may be used for the generation of derivative or variant forms of glycosylation-variant BEHAB using recombinant DNA methodology well known in the art such as, for example, that described in Sambrook et al. (1989, Molecular Cloning: A Laboratory Manual, Cold Spring Harbor Laboratory Press, New York) and Ausubel et al. (1997, Current Protocols in Molecular Biology, Green & Wiley, New York).

Procedures for the introduction of amino acid changes in a protein or polypeptide by altering the DNA sequence encoding the polypeptide are well known in the art and are also described in Sambrook et al. (1989, supra); Ausubel et al. (1997, supra).

The invention includes a nucleic acid encoding a human glycosylation-variant BEHAB wherein a nucleic acid encoding a tag polypeptide is covalently linked thereto. That is, the invention encompasses a chimeric nucleic acid wherein the nucleic acid sequence encoding a tag polypeptide is covalently linked to the nucleic acid encoding a glycosylation-variant BEHAB polypeptide. Such tag polypeptides are well known in the art and include, for instance, green fluorescent protein (GFP), myc, myc-pyruvate kinase (myc-PK), His₆, maltose biding protein (MBP), an influenza virus hemagglutinin tag polypeptide, a flag tag polypeptide (FLAG), and a glutathione-S-transferase (GST) tag polypeptide. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention.

The nucleic acid comprising a nucleic acid encoding a tag polypeptide can be used to localize glycosylation-variant BEHAB within a cell, a tissue, and/or a whole organism (e.g., a mammalian embryo), detect glycosylation-variant BEHAB secreted from a cell, and to study the role(s) of glycosylation-variant BEHAB in a cell. Further, addition of a tag polypeptide facilitates isolation and purification of the “tagged” protein such that the proteins of the invention can be produced and purified readily.

B. Antisense Nucleic Acids

In certain situations, it may be desirable to inhibit expression of BEHAB and the invention therefore includes compositions useful for inhibition of BEHAB expression. Thus, the invention features an isolated nucleic acid complementary to a portion or all of a nucleic acid encoding a mammalian BEHAB molecule which nucleic acid is in an antisense orientation with respect to transcription. Preferably, the antisense nucleic acid is complementary with a nucleic acid having at least about 99.7% homology with SEQ ID NO:7, or a fragment thereof. Preferably, the nucleic acid is about 99.8% homologous, and most preferably, about 99.9% homologous to a nucleic acid complementary to a portion or all of a nucleic acid encoding a mammalian BEHAB having the sequence of SEQ ID NO:7, or a fragment thereof, which is in an antisense orientation with respect to transcription. Most preferably, the nucleic acid is complementary to a portion or all of a nucleic acid that is SEQ ID NO:7, or a fragment thereof. Such antisense nucleic acid serves to inhibit the expression, function, or both, of a BEHAB molecule.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see Cohen, 1989, In: Oligodeoxyribonucleotides, Antisense Inhibitors of Gene Expression, CRC Press, Boca Raton, Fla.; Tullis, 1991, U.S. Pat. No. 5,023,243) incorporated by reference herein in its entirety.

### II. Isolated Polypeptides

The invention also includes an isolated polypeptide comprising a glycosylation-variant BEHAB molecule. Preferably, the nucleic acid is about 1% homologous, more preferably, about 5% homologous, even more preferably about 10% homologous, even more preferably about 20% homologous, even more preferably, the nucleic acid is about 30% homologous, more preferably, about 40% homologous, even more preferably about 50% homologous, even more preferably, the nucleic acid is about 60% homologous, more preferably, about 70% homologous, even more preferably about 80% homologous. Preferably, the nucleic acid is about 90% homologous, more preferably, about 95% homologous, even more preferably about 99% homologous, even more preferably about 99.9% homologous to SEQ ID NO:8, disclosed herein. Even more preferably, the nucleic acid is SEQ ID NO:8. The present invention also provides for analogs of proteins or peptides which comprise a glycosylation-variant BEHAB molecule as disclosed herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention should also be construed to encompass “derivatives,” and “variants” of the peptides of the invention (or of the DNA encoding the same) which derivatives and variants are glycosylation-variant BEHAB peptides which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the mutant BEHAB peptide of the present invention.

The biological/biochemical properties of a glycosylation-variant BEHAB molecule, including a poly-sialyated full-length BEHAB, are disclosed elsewhere herein.

The skilled artisan would understand, based upon the disclosure provided herein, that glycosylation-variant BEHAB biological activity encompasses, but is not limited to, the ability of a molecule or compound to be expressed in glioma, to be detected in glioma, to be expressed on a cell, to be anchored to a cell, and the like.

### III. Vectors

In other related aspects, the invention includes an isolated nucleic acid encoding a glycosylation-variant BEHAB operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

Expression of glycosylation-variant BEHAB, either alone or fused to a detectable tag polypeptide, in cells which either normally express BEHAB, may be accomplished by generating a plasmid, viral, or other type of vector comprising the desired nucleic acid operably linked to a promoter/regulatory sequence which serves to drive expression of the protein, with or without tag, in cells in which the vector is introduced. Many promoter/regulatory sequences useful for driving constitutive expression of a gene are available in the art and include, but are not limited to, for example, the cytomegalovirus immediate early promoter enhancer sequence, the SV40 early promoter, both of which were used in the experiments disclosed herein, as well as the Rous sarcoma virus promoter, and the like. Moreover, inducible and tissue specific expression of the nucleic acid encoding glycosylation-variant BEHAB may be accomplished by placing the nucleic acid encoding glycosylation-variant BEHAB, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Expressing glycosylation-variant BEHAB, including poly-sialyated full-length BEHAB, using a vector allows the isolation of large amounts of recombinantly produced protein.

The invention includes not only methods of producing glycosylation-variant BEHAB, but it also includes methods relating to detecting glycosylation-variant BEHAB expression, protein level, and/or activity since detecting glycosylation-variant BEHAB expression, and/or activity or decreasing glycosylation-variant BEHAB expression and/or activity can be useful in providing effective therapeutics.

Selection of any particular plasmid vector or other DNA vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention thus includes a vector comprising an isolated nucleic acid encoding a human glycosylation-variant BEHAB. The incorporation of a desired nucleic acid into a vector and the choice of vectors is well-known in the art as described in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention also includes cells, viruses, proviruses, and the like, containing such vectors. Methods for producing cells comprising vectors and/or exogenous nucleic acids are well-known in the art. See, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The nucleic acids encoding glycosylation-variant BEHAB, including polysialyated full-length BEHAB may be cloned into various plasmid vectors. However, the present invention should not be construed to be limited to plasmids or to any particular vector. Instead, the present invention should be construed to encompass a wide plethora of vectors which are readily available and/or well-known in the art.

### IV. Recombinant Cells

The invention further includes a method of making a glycosylation-variant BEHAB isoform in a recombinant cell comprising, inter alia, an isolated nucleic acid encoding a BEHAB protein. That is, as demonstrated by the data disclosed herein, a glycosylation-variant BEHAB isoform can be produced in a recombinant cell by transfecting a cell with an isolated nucleic acid encoding BEHAB, or a fragment thereof, and isolating the glycosylation-variant BEHAB isoform therefrom. Cells useful for the production of a glycosylation-variant BEHAB include, for example, an Oli-neu or an U87-MG cell. Further, methods for transfecting a cell and producing a protein therefrom are well known in the art and are described in detail elsewhere herein. Recombinant cells thus include those which express full-length BEHAB, and those that express a glycosylation-variant BEHAB.

Further, it is important to note that the purpose of recombinant cells should not be construed to be limited to the generation of intracranial tumors. Rather, the invention should be construed to include any cell type into which a nucleic acid encoding a glycosylation-variant BEHAB is introduced, including, without limitation, a prokaryotic cell and a eukaryotic cell comprising an isolated nucleic acid encoding glycosylation-variant BEHAB.

The invention includes a eukaryotic cell which, when the recombinant gene of the invention is introduced therein, and the protein encoded by the desired gene is expressed therefrom, where it was not previously present or expressed in the cell or where it is now expressed at a level or under circumstances different than that before the transgene was introduced, a benefit is obtained. Such a benefit may include the fact that there has been provided a system wherein the expression of the desired gene can be studied in vitro in the laboratory or in a mammal in which the cell resides, a system wherein cells comprising the introduced gene can be used as research, diagnostic and therapeutic tools, and a system wherein mammal models are generated which are useful for the development of new diagnostic and therapeutic tools for selected disease states in a mammal.

One of ordinary skill would appreciate, based upon the disclosure provided herein, that a “knock-in” or “knock-out” vector of the invention comprises at least two sequences homologous to two portions of the nucleic acid which is to be replaced or deleted, respectively. The two sequences are homologous with sequences that flank the gene; that is, one sequence is homologous with a region at or near the 5′ portion of the coding sequence of the nucleic acid encoding full-length BEHAB and the other sequence is further downstream from the first. One skilled in the art would appreciate, based upon the disclosure provided herein, that the present invention is not limited to any specific flanking nucleic acid sequences. Instead, the targeting vector may comprise two sequences which remove some or all of, for example, glycosylation-variant BEHAB (i.e., a “knock-out” vector) or which insert (i.e., a “knock-in” vector) a nucleic acid encoding mutant BEHAB, or a fragment thereof, from or into a mammalian genome, respectively. The crucial feature of the targeting vector is that it comprise sufficient portions of two sequences located towards opposite, i.e., 5′ and 3′, ends of the full-length BEHAB open reading frame (ORF) in the case of a “knock-out” vector, to allow deletion/insertion by homologous recombination to occur such that all or a portion of the nucleic acid encoding full-length BEHAB is deleted from a location on a mammalian chromosome.

The design of transgenes and knock-in and knock-out targeting vectors is well-known in the art and is described in standard treatises such as Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York), and the like. The upstream and downstream portions flanking or within the BEHAB coding region to be used in the targeting vector may be easily selected based upon known methods and following the teachings disclosed herein based on the disclosure provided herein including the nucleic and amino acid sequences of glycosylation-variant BEHAB and mutant BEHAB. Armed with these sequences, one of ordinary skill in the art would be able to construct the transgenes and knock-out vectors of the invention.

One skilled in the art would appreciate, based upon this disclosure, that cells comprising decreased levels of glycosylation-variant BEHAB protein, decreased levels of BEHAB and/or BEHAB cleavage product activity, or both, include, but are not limited to, cells expressing inhibitors of BEHAB expression (e.g., antisense or ribozyme molecules, synthetic antibodies or intrabodies).

Methods and compositions useful for maintaining mammalian cells in culture are well known in the art, wherein the mammalian cells are obtained from a mammal including, but not limited to, cells obtained from a mouse, a rat, a human, and the like.

The recombinant cell of the invention can be used to study the effect of qualitative and quantitative alterations in BEHAB levels on tumor progression and invasiveness. This is because the fact that BEHAB is secreted and possesses a hyaluronan binding domain indicates that BEHAB is involved in the function, composition, or activity of the ECM. Further, the recombinant cell can be used to produce mutant BEHAB for use for therapeutic and/or diagnostic purposes. That is, a recombinant cell expressing mutant BEHAB can be used to produce large amounts of purified and isolated mutant BEHAB that can be administered to treat or alleviate a disease, disorder or condition associated with or caused by BEHAB expression, activity, and/or cleavage.

Alternatively, recombinant cells expressing mutant BEHAB can be administered in ex vivo and in vivo therapies where administering the recombinant cells thereby administers the protein to a cell, a tissue, and/or a mammal. Additionally, the recombinant cells are useful for the discovery of BEHAB receptor and BEHAB signaling pathways.

### V. Antibodies

Also included is an antibody that specifically binds BEHAB, a glycosylation-variant BEHAB, or fragments thereof.

The skilled artisan, when equipped with the present disclosure, would also understand that the present invention further comprises antibodies that bind a glycosylation-variant BEHAB isoform, including an underglycosylated BEHAB isoform and an unglycosylated BEHAB isoform. The generation of antibodies is described elsewhere herein, and their production is accomplished using techniques and skills well known in the art. Antibodies that bind glycosylation-variant BEHAB, including underglycosylated BEHAB and unglycosylated BEHAB include, but are not limited to the B5, B6 and B_{CRP} antibodies described in the experimental details herein and elsewhere in the art (Matthews et al., 2000, J. Biol. Chem. 275: 22695-22703). Further, the antibodies described herein can bind various forms of mammalian BEHAB, including rat and human, and art thus useful in the present invention for the detection, diagnosis, and treatment of primary CNS tumors associated with BEHAB.

The present invention is not limited to the antibodies enumerated herein, but rather also includes anti-glycosylation-variant BEHAB antibodies discovered and generated in the future. An antibody to a glycosylation-variant BEHAB, including a differently-glycosylated, underglycosylated and unglycosylated BEHAB, can be generated in a variety of ways well known in the art. As a non-limiting example, a nucleic acid encoding BEHAB, or a fragment thereof, can be transformed into an organism that does not glycosylate the proteins it produces, such as E. coli. Methods for the production of proteins in E. coli and other prokaryotic species are well known in the art and are described elsewhere herein. The protein isolated from a non-glycosylating prokaryotic species can then be administered to a mammal to generate antibodies, as is described herein. The antibodies specifically bind a glycosylation-variant BEHAB isoform, including unglycosylated BEHAB and polysialyated full-length BEHAB.

Further, antibodies to glycosylation-variant BEHAB can be generated by contacting a full-length BEHAB protein with glycosidases in order to remove some or all of the sugars and carbohydrates associated with the BEHAB protein backbone. Such glycosidases are well known in the art, and a number of relevant glycosidases are described elsewhere herein. Further, the skilled artisan, when equipped with the present disclosure and the data disclosed herein, would readily be able to select specific glycosidases for the removal of a certain family of sugars or carbohydrates while optionally retaining others on sugars and carbohydrates on the BEHAB molecule. The BEHAB molecule, after treatment with a glycosidase, can then be administered to an animal for the generation of antibodies to glycosylation-variant-BEHAB. Methods for the administration of a protein to a mammal and the generation of an antibody are well known in the art and are described herein.

The invention further comprises generating antibodies specific to glycosylation-variant BEHAB. Such antibodies are useful in the compositions, methods and kits disclosed elsewhere herein. As a non-limiting example, an antibody specific to glycosylation-variant BEHAB can be generated by administering a peptide or protein comprising fragments of the primary amino acid sequence of BEHAB. Such fragments can comprise consensus glycosylation sites present in the primary amino acid sequence of BEHAB. The skilled artisan will readily recognize such consensus glycosylation sites by their sequences and amino acid content. As an example, O-linked saccharides are usually attached via a glycosidic bond on a threonine or serine residue, and in some cases, on hydroxylysine or hydroxyproline. Further, N-linked saccharides are often attached to an asparagine residue, often at a site having a sequence of any amino acid bound to an asparagine bound to any amino acid bound to threonine. Thus, the skilled routineer, when armed with the present disclosure and the methods disclosed herein, would readily be able to identify consensus glycosylation sites in a BEHAB primary amino acid sequence, generate peptides for immunizing an animal comprising these consensus glycosylation sites, and generate antibodies that specifically bind glycosylation-variant BEHAB. Such antibodies are useful in therapeutic treatments, including, but not limited to immunizing a mammal against the formation of primary CNS tumors, treating a primary CNS tumor, detecting a primary CNS tumor in a mammal either in vivo or in vitro, and other methods and uses disclosed elsewhere herein.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the BEHAB portion is rendered immunogenic (e.g., BEHAB conjugated with keyhole limpet hemocyanin, KLH) and a portion comprising the respective rodent and/or human BEHAB amino acid residues. The chimeric proteins are produced by cloning the appropriate nucleic acids encoding BEHAB (e.g., SEQ ID NO:5 into a plasmid vector suitable for this purpose, such as but not limited to, pMAL-2 or pCMX. Other methods of producing antibodies that specifically bind BEHAB and portions thereof are detailed in Matthews et al. (2000, J. Biol. Chem. 275: 22695-22703).

However, the invention should not be construed as being limited solely to polyclonal antibodies that bind a full-length BEHAB. Rather, the invention should be construed to include other antibodies, as that term is defined elsewhere herein, to mammalian BEHAB, or portions thereof. Further, the present invention should be construed to encompass antibodies that, among other things, bind to BEHAB and are able to bind BEHAB present on Western blots, in immunohistochemical staining of tissues thereby localizing BEHAB in the tissues, and in immunofluorescence microscopy of a cell transiently or stably transfected with a nucleic acid encoding at least a portion of BEHAB.

One skilled in the art would appreciate, based upon the disclosure provided herein, that the antibody can specifically bind with any portion of the protein and the full-length protein can be used to generate antibodies specific therefor. However, the present invention is not limited to using the full-length protein as an immunogen. Rather, the present invention includes using an immunogenic portion of the protein to produce an antibody that specifically binds with mammalian BEHAB. That is, the invention includes immunizing an animal using an immunogenic portion, or antigenic determinant, of the BEHAB protein, for example, the epitope comprising the cleavage site, or a new antigenic site produced by proteolytic cleavage.

The antibodies can be produced by immunizing an animal such as, but not limited to, a rabbit or a mouse, with a BEHAB protein, or a portion thereof, or by immunizing an animal using a protein comprising at least a portion of BEHAB, or a fusion protein including a tag polypeptide portion comprising, for example, a maltose binding protein tag polypeptide portion, covalently linked with a portion comprising the appropriate BEHAB amino acid residues. One skilled in the art would appreciate, based upon the disclosure provided herein, that smaller fragments of these proteins can also be used to produce antibodies that specifically bind BEHAB.

One skilled in the art would appreciate, based upon the disclosure provided herein, that various portions of an isolated BEHAB polypeptide can be used to generate antibodies to either epitopes comprising the cleavage site of BEHAB or to epitopes present on the cleavage products of BEHAB. Once armed with the sequence of BEHAB and the detailed analysis localizing the various epitopes and cleavage products of the protein, the skilled artisan would understand, based upon the disclosure provided herein, how to obtain antibodies specific for the various portions of a mammalian. BEHAB polypeptide using methods well-known in the art or to be developed.

Therefore, the skilled artisan would appreciate, based upon the disclosure provided herein, that the present invention encompasses antibodies that neutralize and/or inhibit BEHAB activity (e.g., by inhibiting necessary BEHAB cleavage product receptor/ligand interactions or BEHAB cleavage) which antibodies can recognize BEHAB or BEHAB cleavage products.

The invention should not be construed as being limited solely to the antibodies disclosed herein or to any particular immunogenic portion of the proteins of the invention. Rather, the invention should be construed to include other antibodies, as that term is defined elsewhere herein, to BEHAB, or portions thereof, or to proteins sharing at least some homology with a polypeptide having the amino acid sequence of SEQ ID NO:8. Preferably, the polypeptide is about 1% homologous, more preferably, about 5% homologous, more preferably, about 10% homologous, even more preferably, about 20% homologous, more preferably, about 30% homologous, preferably, about 40% homologous, more preferably, about 50% homologous, even more preferably, about 60% homologous, more preferably, about 70% homologous, even more preferably, about 80% homologous, preferably, about 90% homologous, more preferably, about 95% homologous, even more preferably, about 99% homologous, and most preferably, about 99.9% homologous to human BEHAB (SEQ ID NO:8).

One skilled in the art would appreciate, based upon the disclosure provided herein, that the antibodies can be used to localize the relevant protein in a cell and to study the role(s) of the antigen recognized thereby in cell processes. Moreover, the antibodies can be used to detect and or measure the amount of protein present in a biological sample using well-known methods such as, but not limited to, Western blotting and enzyme-linked immunosorbent assay (ELISA). Moreover, the antibodies can be used to immunoprecipitate and/or immuno-affinity purify their cognate antigen using methods well-known in the art. In addition, the antibody can be used to decrease the level of BEHAB or BEHAB cleavage products in a cell thereby inhibiting the effect(s) of BEHAB or BEHAB cleavage products in a cell. Thus, by administering the antibody to a cell or to the tissues of a mammal or to the mammal itself, the required BEHAB receptor/ligand interactions are therefore inhibited such that the effect of BEHAB cleavage is also inhibited. One skilled in the art would understand that inhibiting BEHAB cleavage using an anti-BEHAB antibody can include, but is not limited to, decreased tumor size, increased survival, and the like.

One skilled in the art would appreciate, based upon the disclosure provided herein, that the invention encompasses administering an antibody that specifically binds with BEHAB orally, parenterally, intraventricularly, intrathecally, intraparenchymally or by multiple routes, to inhibit BEHAB cleavage in the brain. Administration can include delivery by bioengineered polymers, direct injection, through an Ommaya reservoir (A device implanted under the scalp that is used to deliver anticancer drugs to the cerebrospinal fluid, or other such means well known to one of skill in the art of neurosurgery.

The invention encompasses polyclonal, monoclonal, synthetic antibodies, and the like. One skilled in the art would understand, based upon the disclosure provided herein, that the crucial feature of the antibody of the invention is that the antibody bind specifically with BEHAB. That is, the antibody of the invention recognizes BEHAB, or a fragment thereof (e.g., an immunogenic portion or antigenic determinant thereof), on Western blots, in immunostaining of cells, and immunoprecipitates BEHAB using standard methods well-known in the art.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein.

Further, the antibody of the invention may be “humanized” using the technology described in, for example, Wright et al. (1992, Critical Rev. Immunol. 12:125-168), and in the references cited therein, and in Gu et al. (1997, Thrombosis and Hematocyst 77:755-759). The present invention also includes the use of humanized antibodies specifically reactive with epitopes of BEHAB. Such antibodies are capable of specifically binding BEHAB, or a fragment thereof. The humanized antibodies of the invention have a human framework and have one or more complementarity determining regions (CDRs) from an antibody, typically, but not limited to a mouse antibody, specifically reactive with BEHAB, or a fragment thereof. Thus, for example, humanized antibodies to BEHAB are useful in the treatment of primary CNS tumors such as gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, and the like.

When the antibody used in the invention is humanized, the antibody may be generated as described in Queen, et al. (U.S. Pat. No. 6,180,370), Wright et al., (1992, Critical Rev. Immunol. 12:125-168) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as BEHAB, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671, which is herein incorporated by reference. CDRs useful in producing the antibodies of the present invention may be similarly derived from DNA encoding monoclonal antibodies capable of binding to BEHAB. Such humanized antibodies may be generated using well known methods in any convenient mammalian source capable of producing antibodies, including, but not limited to, mice, rats, rabbits, or other vertebrates. Suitable cells for constant region and framework DNA sequences and host cells in which the antibodies are expressed and secreted, can be obtained from a number of sources, for example, American Type Culture Collection, Manassas, Va.

In addition to the humanized antibodies discussed above, other modifications to native antibody sequences can be readily designed and manufactured utilizing various recombinant DNA techniques well known to those skilled in the art. Moreover, a variety of different human framework regions may be used singly or in combination as a basis for humanizing antibodies directed to BEHAB, including glycosylation-variant BEHAB and poly-sialyated full-length BEHAB. In general, modifications of genes may be readily accomplished using a variety of well-known techniques, such as site-directed mutagenesis (Gillman and Smith, Gene, 8:81-97 (1979); Roberts et al., 1987, Nature, 328:731-734).

Alternatively, a phage antibody library may be generated. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor Laboratory, New York).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (992, Critical Rev. Immunol. 12:125-168).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

### VI. Compositions

The present invention encompasses a glycosylation-variant BEHAB isoform, including, but not limited to differently-glycosylated, underglycosylated BEHAB, poly-sialyated glycosylation variant BEHAB and unglycosylated BEHAB. The glycosylation-variant BEHAB of the present invention comprises a BEHAB molecule with altered or less than the full complement of sugars and carbohydrates found on full-length BEHAB. As disclosed by the data herein, glycosylation-variant BEHAB is the major upregulated form of BEHAB in primary CNS tumors, including, but not limited to, gliomas. Thus the present invention includes a glycosylation-variant BEHAB that is useful for, inter alia, a diagnostic tool for primary CNS tumors, a research tool for elucidating the interaction of the neural extracellular matrix with cancer-causing mutations, dysfunctions, and the like. Further, the glycosylation-variant BEHAB of the present invention is useful as a reagent in compositions, methods and kits for the detection, treatment, and diagnosis of primary CNS tumors, including, but not limited to immunotherapy of primary CNS tumors, such as glioma.

Glycosylation-variant BEHAB can be made according to the methods disclosed herein. That is, the present invention comprises methods for the isolation of glycosylation-variant BEHAB from the particulate fraction of brain homogenate, and further includes methods for the differentiation of glycosylation-variant BEHAB from other BEHAB molecules, including full-length BEHAB and GPI-linked BEHAB.

The present invention further comprises methods for the generation of glycosylation-variant BEHAB in a recombinant cell. That is, the skilled artisan, when equipped with the present disclosure and the data herein, can produce glycosylation-variant BEHAB by transfecting a cell with an isolated nucleic acid encoding BEHAB, or a fragment thereof, and isolating glycosylation-variant BEHAB from a cell. Isolated nucleic acids for this purpose are disclosed elsewhere herein, as are methods for the transfection and expression of a protein in a cell. Preferably, the cell is a cell that expresses glycosylation-variant BEHAB, such as, but not limited to, and Oli-neu cell.

As described by the data disclosed herein, a glycosylation-variant BEHAB can be differentiated from full-length BEHAB or GPI-anchored BEHAB through various methods. Such methods include SDS-PAGE electrophoresis, immunofluorescence and localization, immunoprecipitation, and the like. Further, the skilled artisan would readily be able to distinguish between a different isoform of a protein based on glycosylation using techniques known in the art and described herein.

### VII. Methods

A. Methods of Treating a Primary CNS Tumor

The present invention is based, in part, on the novel discovery that BEHAB plays a significant role in primary CNS tumor progression, invasiveness and the survival time of mammals with brain tumors. As demonstrated by the data disclosed herein, BEHAB cleavage potentiates the progression of primary CNS tumors, and inhibition of cleavage, and/or inhibition of the function of BEHAB and its cleavage products can be used as a treatment for a primary CNS tumor in a mammal. In all instances, whether treating or diagnosing a primary CNS tumor, the most preferred mammal is a human.

The present invention includes a method of treating a primary CNS tumor in a mammal, preferably a human. This is because, as demonstrated by the data disclosed elsewhere herein, cleavage of BEHAB, and/or the function, biological activity and expression of BEHAB cleavage products is critical to the progression and invasiveness of primary CNS tumors. Therefore, as is evident from the data presented herein, inhibiting the cleavage of BEHAB, and/or inhibiting the function, biological activity, and expression of BEHAB cleavage products can serve as a treatment for primary CNS tumors. One skilled in the art would appreciate, based on the present disclosure, that inhibiting the cleavage of BEHAB provides an important and novel therapeutic for the treatment of among other things, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, reactive gliosis, and the like. One of skill in the art will also recognize that, like primary CNS tumors, glial cell activation and proliferation arises from injury to the CNS, i.e. the brain and spinal cord. Such glial cell activation and proliferation that result from these injuries are well known in the art and often referred to as reactive gliosis. Reactive gliosis is detailed in, for example, Streit (2000, Toxicol. Pathol., 28:28-30). The present invention includes methods for treating reactive gliosis in a mammal, preferably a human.

An inhibitor of BEHAB cleavage is administered to a mammal, thereby decreasing BEHAB cleavage and providing a therapeutic benefit. The skilled artisan would appreciate, based upon the disclosure provided herein, that BEHAB cleavage can be inhibited using a wide range of techniques known or to be developed in the future. That is, the invention encompasses inhibiting the cleavage of BEHAB in a mammal, and thereby preventing the progression and invasiveness of a primary CNS tumor. The present invention discloses methods for inhibiting BEHAB cleavage in a mammal, e.g. blocking the cleavage site, titrating the protease responsible for cleavage, and expressing or administering a non-cleavable BEHAB mutant. This is because, as demonstrated by the data disclosed herein, affecting the cleavage of BEHAB mediates a variety of effects, including, but not limited to decreased tumor size and increased survival time in mammals afflicted with primary CNS tumors, and thereby provides a novel and powerful therapeutic for primary CNS tumors.

The skilled artisan will further understand when equipped with this disclosure and the data presented herein, that administering to a mammal an inhibitor of the function, biological activity, and expression of BEHAB and/or its cleavage products provides a beneficial therapeutic to a mammal with a primary CNS tumor. The present invention includes methods for reducing or preventing the expression of BEHAB and binding the cleavage products and/or their ligands. As demonstrated by the data disclosed herein, increased levels of BEHAB and the biological activity of BEHAB cleavage products mediate enhanced progression of primary CNS tumors, resulting in decreased survival rates and larger tumors. Therefore, a method for inhibiting BEHAB expression or BEHAB cleavage product expression and/or function is included in the present invention.

The skilled artisan would understand that inhibiting BEHAB cleavage encompasses blocking the cleavage site, titrating the protease responsible for cleavage, and expressing and/or administering a non-cleavable BEHAB mutant. The present invention includes a method for inhibiting the cleavage of BEHAB by blocking the cleavage site on the protein. As disclosed herein, the cleavage site comprises Glu³⁹⁵-Ser³⁹⁶ of the BEHAB protein. Therefore, inaccessibility of this cleavage site to a protease can prevent the cleavage of BEHAB. The present invention therefore includes methods for inhibiting the cleavage of BEHAB by blocking access to the cleavage site by proteases. As an example, an antibody or other ligand to a portion of the protein comprising the cleavage site, or a peptide or a small molecule that interacts with the cleavage site, would block access to the protein by a protease, thereby inhibiting BEHAB cleavage. The skilled artisan would appreciate, when armed with the disclosure and data disclosed herein, that an antibody can specifically bind a short peptide comprising the cleavage site, or to a larger portion of the BEHAB protein, provided that the antibody or the ligand blocks the cleavage site.

Methods of generating antibodies to BEHAB are well known in the art (Matthews et al., 2000, J. Biol. Chem. 275: 22695-22703) and are disclosed elsewhere herein. Further, methods for producing antibodies that specifically bind certain epitopes of a protein are well known in the art and can be accomplished using standard methods disclosed herein and elsewhere, see Harlow et al. (1988, Antibodies: A Laboratory Manual, Cold Spring Harbor, N.Y.).

One of skill in the art will appreciate that an antibody can be administered as a protein, a nucleic acid construct encoding a protein, or both. Numerous vectors and other compositions and methods disclosed elsewhere herein are well known for administering a protein or a nucleic acid construct encoding a protein to cells or tissues. Therefore, the invention includes a method of administering an antibody or nucleic acid encoding an antibody (synthetic antibody) that is specific for BEHAB (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

One skilled in the art would understand, based upon the disclosure provided herein, that an antibody can be administered such that it blocks the cleavage site on BEHAB present in a mammal. Moreover, the invention encompasses administering an antibody that specifically binds with BEHAB, or a nucleic acid encoding the antibody, wherein the molecule further comprises an intracellular retention sequence such that the antibody binds with BEHAB and prevents its GPI-anchored expression or secretion. Such antibodies, frequently referred to as “intrabodies”, are well known in the art and are described in, for example, Marasco et al. (U.S. Pat. No. 6,004,490) and Beerli et al. (1996, Breast Cancer Research and Treatment 38:11-17). Thus, the invention encompasses methods comprising inhibiting BEHAB cleavage where BEHAB is present in a mammal, as well as methods of inhibiting BEHAB cleavage comprising inhibiting BEHAB being present in its GPI-anchored on a cell membrane form or its secreted, and such methods as become known in the future.

The present invention further comprises a method of treating a primary CNS tumor or reactive gliosis in a mammal, including a human, by administering to the mammal an effective amount of glycosylation-variant BEHAB isoform inhibitor. That is, the present invention encompasses a method for treating a primary CNS tumor in a mammal, including, gliomas, well-differentiated astrocytomas, anaplastic astrocytomas, glioblastoma multiforme, ependymomas, oligodendrogliomas, ganglioneuromas, mixed gliomas, brain stem gliomas, optic nerve gliomas, meningiomas, pineal tumors, pituitary tumors, pituitary adenomas, primitive neuroectodermal tumors, schwannomas, vascular tumors, lymphomas, and the like. The method comprises administering an antibody to a mammal wherein the antibody or other ligand binds to a glycosylation-variant BEHAB isoform and thus treats a primary CNS tumor. This is because, as demonstrated by the data disclosed herein, glycosylation-variant BEHAB is the major isoform of BEHAB present in primary CNS tumors, including gliomas and the like. Therefore, the present invention is useful in inhibiting the activity of a glycosylation-variant BEHAB in the CNS and thus treating a primary CNS tumor.

Methods for the generation and administration of an antibody that specifically binds a glycosylation-variant BEHAB isoform are well known in the art and are described elsewhere herein. The present invention further comprises intrabodies, antibodies administered as a protein, and antibodies administered as a nucleic acid construct encoding an antibody that binds a glycosylation-variant BEHAB isoform, including an underglycosylated BEHAB isoform and an unglycosylated BEHAB isoform.

The present invention also encompasses methods for inhibiting BEHAB cleavage by inhibiting the protease responsible for BEHAB cleavage. This is because, as is evident from the data presented herein, BEHAB is cleaved by a protease at a specific site, but inhibiting the cleavage of BEHAB results in, among other things, smaller tumor volumes and increased animal survival rates. Therefore, the present invention includes a method of inhibiting BEHAB cleavage by inhibiting the protease that cleaves BEHAB.

One of skill in the art will recognize that inhibiting a protease comprises administering to a mammal an effective amount of a protease inhibitor. Such inhibitors include, but are not limited to, chemical compounds, including tissue inhibitor of metalloproteinases 2, tissue inhibitor of metalloproteinases 3, inhibitors of ADAMTS proteases, small molecules, an antibody or other molecule that specifically binds a protease that cleaves BEHAB, and the like. Specific protease inhibitors are well known in the art, and are discussed in, for example, Martel-Pelletier et al., (2001, Best Pract. Res. Clin. Rheumatol. 15:805-29). The skilled artisan, when armed with the present disclosure and teachings herein, will readily understand how to administer a protease inhibitor to a mammal, and therefore, the present invention encompasses protease inhibitors as a treatment for primary CNS tumors.

The present invention also encompasses methods for inhibiting BEHAB cleavage by titrating the protease responsible for BEHAB cleavage. This is because, as is evident from the data presented herein, BEHAB is cleaved by a protease at a specific site, but the mutant BEHAB of the present invention cannot be cleaved by a protease, as measured in both in vivo and in vitro assays. Further, the protease that cleaves BEHAB is present in the body in limited amounts and limited locations compared to other metalloproteinases. Therefore, an uncleavable BEHAB is capable of titrating the protease so it is not available to cleave endogenous BEHAB. One of skill in the art will recognize that titrating a protease encompasses providing a substrate that reduces the functional concentration of the protease in a mammal, preferably a human, that is available to cleave BEHAB. Titrating a protease further includes providing a substrate that is recognized and bound by a protease, resulting in a decline in the number of proteases or protease active sites available to cleave BEHAB.

As described more fully elsewhere herein, a tumor expressing a mutant, uncleavable form of BEHAB, even in the presence of endogenous BEHAB, results in among other things, smaller tumor volumes and increase survival rates in animals. These data indicate that even though endogenous BEHAB is present in a cell, the additional presence of an uncleavable BEHAB results in the decreased progression of a primary CNS tumor in an art accepted in vivo primary CNS tumor model. The data further indicate that when tumors expressing exogenous mutant BEHAB are compared to tumors expressing exogenous full-length BEHAB, tumors expressing mutant BEHAB are both smaller and result in longer animal survival times. While not wishing to be bound by any particular theory, the data presented herein indicate that an uncleavable BEHAB mutant titrates the protease responsible for BEHAB cleavage, and as a result of decreased cleavage, decreased tumor progression ensues.

The skilled artisan would appreciate, based on the present disclosure and the data disclosed herein that a non-cleavable substrate for the protease inhibits tumor progression by decreasing tumor size and increasing survival rates in animals afflicted with primary CNS tumors. Therefore, the present invention includes a method for treating a primary CNS tumor by titrating the protease that cleaves BEHAB.

Compounds used to titrate the protease that cleaves BEHAB include, but are not limited to, peptides, proteins, mimetopes and peptidomimetics. As disclosed elsewhere herein, non-cleavable BEHAB (mutant BEHAB, SEQ ID NO:3) comprises the native BEHAB protein with a mutation in the amino acid sequence surrounding the cleavage site, specifically a mutation of Glu-Ser-Glu-Ser-Arg-Gly to Glu-Ser-Glu-Asn-Val-Tyr (SEQ ID NO:1 and SEQ ID NO:2, respectively). One of skill in the art will readily appreciate that a peptide derived from full length mutant BEHAB can exhibit the same protease titrating properties as the full length mutant BEHAB protein set forth in SEQ ID NO:3. Thereby the present invention encompasses the full length mutant BEHAB protein and truncated mutant BEHAB peptides comprising protease titrating activity.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The peptides of the present invention may be readily prepared by standard, well-established solid-phase peptide synthesis (SPPS) as described by Stewart et al. (Solid Phase Peptide Synthesis, 2nd Edition, 1984, Pierce Chemical Company, Rockford, Ill.) and as described by Bodanszky and Bodanszky (The Practice of Peptide Synthesis, 1984, Springer-Verlag, New York). At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenyl esters.

Examples of solid phase peptide synthesis methods include the BOC method which utilized tert-butyloxycarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF (hydrofluoric acid) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequencers, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies or for specific uses. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

One of skill in the art would readily appreciate that a mutant BEHAB protein or peptide capable of titrating a protease that cleaves BEHAB may be administered to a mammal as an isolated nucleic acid encoding a mutant BEHAB protein or peptide. Methods of expressing a desired protein in a cell or a mammal are well known in the art, and when combined with the present disclosure and the data herein, the skilled artisan will to be able to express a mutant BEHAB protein or peptide in a cell or a mammal without undue experimentation.

One of skill in the art will appreciate that many methods exist for the expression of a protein or peptide in a cell or a mammal, including the introduction of a vector or expression vector comprising an isolated nucleic acid encoding the desired protein or peptide into a cell or mammal. The skilled artisan will further appreciate that a vector can comprise the isolated nucleic of SEQ ID NO:4, or some biologically active portion thereof.

The present invention also includes mimetopes of a mutant BEHAB protein and peptide of the present invention. As used herein, a mimetope of a mutant BEHAB protein or peptide refers to any compound that is able to mimic the activity of such a mutant BEHAB protein or peptide (e.g., ability to titrate a protease that cleaves BEHAB, thereby preventing the cleavage of native BEHAB), often because the mimetope has a structure that mimics the mutant BEHAB protein or peptide. It is to be noted, however, that the mimetope need not have a structure similar to an mutant BEHAB protein or peptide as long as the mimetope functionally mimics the protein. Mimetopes can be, but are not limited to: peptides that have been modified to decrease their susceptibility to degradation; anti-idiotypic and/or catalytic antibodies, or fragments thereof, non-proteinaceous immunogenic portions of an isolated protein (e.g., carbohydrate structures); synthetic or natural organic or inorganic molecules, including nucleic acids; and/or any other peptidomimetic compounds. Mimetopes of the present invention can be designed using computer-generated structures of a mutant BEHAB protein or peptide of the present invention. Mimetopes can also be obtained by generating random samples of molecules, such as oligonucleotides, peptides or other organic molecules, and screening such samples by affinity chromatography techniques using the corresponding binding partner, (e.g., a protease that cleaves BEHAB or anti-BEHAB antibody). A preferred mimetope is a peptidomimetic compound that is structurally and/or functionally similar to a mutant BEHAB protein or peptide of the present invention, particularly to the cleavage site of the mutant BEHAB protein. Methods for generating mimetopes and peptidomimetics are well known in the art, and are detailed in, for example, Kazmierski (1999, Peptidomimetics Protocols (Methods in Molecular Medicine Vol. 23) Humana Press, Totowa N.J.).

The present invention also includes methods for inhibiting the expression and/or activity of BEHAB in a mammal. The skilled artisan will understand, when equipped with the present disclosure and the data disclosed herein, that higher levels of BEHAB expression increase tumor size and decrease survival rates in mammals afflicted with primary CNS tumors. That is, the data presented elsewhere herein demonstrate, for the first time, that mammals with primary CNS tumors overexpressing BEHAB have larger tumor volumes and shorter survival times when compared to mammals expressing normal levels of BEHAB, or to mammals expressing mutant BEHAB. Thus, the skilled artisan will certainly appreciate that a method of treating a primary CNS tumor encompasses inhibiting BEHAB expression.

An inhibitor of BEHAB expression and/or activity is administered to a mammal thereby decreasing BEHAB and providing a therapeutic benefit. The skilled artisan would appreciate, based upon the disclosure provided herein, that BEHAB can be inhibited using a wide plethora of techniques well-known in the art or to be developed in the future. That is, the invention encompasses inhibiting BEHAB expression, e.g., inhibition of transcription and/or translation. This is because, as demonstrated by the data disclosed elsewhere herein, reduced levels of BEHAB expression and/or activity mediated a variety of effects, including, but not limited to, decreased tumor size and increased survival rates. Thus, inhibiting BEHAB includes, but is not limited to, inhibiting translation and/or transcription of a nucleic acid encoding the protein.

Further, the routineer would understand, based upon the disclosure provided elsewhere herein, that inhibition of BEHAB includes, but is not limited to, inhibiting the biological activity of the molecule. This is because, as the data disclosed elsewhere herein demonstrate, inhibition of BEHAB activity, in that BEHAB is not cleaved by an endogenous protease, limits the progression of a primary CNS tumor. These data indicate that inhibition of BEHAB activity provides a therapeutic benefit for treatment of a disease, such as, but not limited to, primary CNS tumors, and the like.

The present invention encompasses inhibiting BEHAB by inhibiting expression of a nucleic acid encoding BEHAB. Methods for inhibiting the expression of a gene are well known to those of ordinary skill in the art, and include the use of ribozymes or antisense nucleic acid molecules.

Antisense nucleic acid molecules are DNA or RNA molecules that are complementary to some portion of an mRNA molecule. When present in a cell, anti sense nucleic acids hybridize to an existing mRNA molecule and inhibit translation into a gene product. Inhibiting the expression of a gene using an antisense nucleic acid molecule is well known in the art (Marcus-Sekura, 1988, Anal. Biochem. 172:289), as are methods to express an antisense nucleic acid molecule in a cell (Inoue, 1993, U.S. Pat. No. 5,190,931).

The invention encompasses inhibiting the expression of BEHAB using a ribozyme. Using ribozymes for inhibiting gene expression is well known to those of ordinary skill in the art (Cech et al., 1992, J. Biol. Chem. 267:17479-17482; Hampel et al., 1989, Biochemistry 28: 4929-4933; Altman et al., 1992, U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030-3034), allowing the selective cleavage of specific mRNA molecules. Given the nucleotide sequence of BEHAB is well known in the art (Hockfield et al., 1997, U.S. Pat. No. 5,635,370) one of ordinary skill in the art can synthesize an antisense polynucleotide or ribozyme without undue experimentation, provided with the disclosure and references incorporated herein.

The skilled artisan will further appreciate, when armed with the present disclosure and the data presented herein, that cleavage of BEHAB mediates progression of primary CNS tumors. While not wishing to be bound by any particular theory, it can be theorized that while BEHAB is normally expressed endogenously at low levels and does not necessarily cause primary CNS tumors during normal expression, the cleavage of BEHAB, or more specifically the products of the cleavage event mediate the progression of a primary CNS tumor in a mammal. Thereby, as will be recognized by one of skill in the art, inhibiting the activity of the BEHAB cleavage products can be used as a method of treating a mammal afflicted with a primary CNS tumor.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

The materials and methods used in the experiments presented in this Example are now described.

### Human Tissue:

All studies regarding samples of human tissue were performed in compliance with the guidelines of the Human Investigations Committee at Yale University School of Medicine. Fresh-frozen surgical samples of intracranial tumors, including glioma, meningioma, epidermoid tumor, schwannoma and medulloblastoma were obtained from Yale-New Haven Medical Hospital (New Haven Conn.). Human glioma samples (8 female, 13 male) were independently graded as previously described (Jaworski et al., 1996, Cancer Res, 56: 2293-2298). Postmortem brain samples (10 female, 10 male, with postmortem interval ranging from 2 to 31 hours) from individuals who had died without neurological pathologies or complications served as controls for the normal level of BEHAB protein expression in normal human cortex. Samples were obtained from the Brain and Tissue Banks for Developmental Disorders (University of Maryland, Baltimore Md.). Fresh-frozen surgical samples of epilepsy foci were kindly provided by Dr. D. Spencer (Department of Neurosurgery, Yale University Medical School). Postmortem brain samples from individuals diagnosed with Alzheimer's disease were kindly provided by Dr. G. W. Rebeck (Department of Neuroscience, Georgetown University, Washington D.C.). All samples were stored at −70° C. until further processing.

### Subcellular Fractionation:

Brain and tumor samples were quickly thawed on ice and homogenized in 10 volumes of 25 mM Tris HCl, pH 7.4, containing 0.32 M sucrose (TS buffer) and a protease inhibitor cocktail (Complete, EDTA-free, Roche, Nutley, N.J.). The homogenate was centrifuged at 950 g×10 minutes and the nuclear pellet (P1) was washed once by rapid rehomogenization in TS buffer and centrifuged as above. Post-nuclear supernatants were combined and centrifuged at 100,000 g×60 minutes to provide total particulate (membrane-enriched) and soluble fractions. Aliquots of the subcellular fractions were equilibrated at a final total protein concentration of 1-2 mg/ml in CH buffer (40 mM Tris HCl, 40 mM sodium acetate, pH 8.0), containing 10 mM EDTA, and treated with 0.25 U/ml of protease-free chondroitinase ABC from Proteus vulgaris (EC 4.2.2.4, Seikagaku, East Fallmouth, Mass.) for 8 h at 37° C. Chondroitinase activity was stopped by boiling the samples in the presence of 1× gel-loading buffer.

Release of BEHAB/Brevican Isoforms from Brain Membranes:

To characterize the association of different BEHAB isoforms with the cell membrane, total membranes (˜1 mg total protein/ml) obtained from control and glioma samples were resuspended in 50 mM Tris HCl buffer, pH 7.4, in the presence or absence of 10 mM EDTA for 1 hour at 4° C. Alternatively, membranes were resuspended in 100 mM sodium carbonate, pH 11.3, for 30 minutes at 4° C. After incubation, membranes were centrifuged at 20,800 g for 20 minutes. Released BEHAB was recovered in the supernatant, and the membranes containing retained BEHAB were washed twice with 50 mM Tris HCl buffer and resuspended in the same initial volume. All samples were finally equilibrated with CH buffer and treated with chondroitinase ABC prior to protein electrophoresis. For immunoprecipitation studies, membranes were first extracted for 1 hour at 4° C. in 50 mM Tris HCl, pH 7.4, containing 300 mM NaCl and 0.6% w/v CHAPS. Solubilized proteins were immunoprecipitated with the rabbit polyclonal anti-BEHAB antibody B6, described elsewhere herein, preadsorbed to protein A-sepharose (Amersham-Pharmacia Biotech, Piscataway, N.J.), according to standard protocols known in the art.

### Cell Cultures and Transfections:

The human glioma cell line U87-MG (American Type Culture Collection, Manassas, Va.) was grown at 5% CO₂ in DMEM medium (Gibco, Gaithersburg, Md.) supplemented with 10% FCS (Hyclone, Logan Utah), 50 μg/ml penicillin and 50 μg/ml streptomycin (Gibco, Gaithersburg, Md.). A clone comprising the complete coding sequence of human BEHAB (GenBank Accession No. BC010571, nucleotides 1-3245) was purchased from Invitrogen (La Jolla, Calif.) and subcloned from the original pSPORT6.1 plasmid into the EcoR1-Not1 restriction sites of a pcDNA3.1(+) plasmid (Invitrogen, La Jolla, Calif.). Cells were transfected employing Lipofectamine 2000 (Invitrogen, La Jolla, Calif.) at a ratio of Lipofectamine (μl):DNA (μg) of 2:1 according to the manufacturers protocol. Control transfections were performed with the parental pcDNA3.1 (+) vector.

### Preparation of Cell Membranes and Immunocytochemistry:

Cells were routinely changed to serum-free medium Optimem (Gibco, Gaithersburg, Md.) 24 hours post-transfection and collected 24 hours after the medium change. Collected cells were lysed in 25 mM phosphate buffer, pH 7.4, containing a protease inhibitor cocktail (Complete, EDTA-free, Roche, Nutley, N.J.) and 2 U/ml RNAse-free DNAse I (Roche, Nutley, N.J.). Total membranes were obtained by centrifugation at 20,800 g×30 minutes and prepared for protein electrophoresis. Culture medium was concentrated by ultradiafiltration and equally processed for SDS-PAGE.

For live immunocytochemical staining of transfected U87-MG cells, cultures were grown on poly-L-lysine (10 μg/ml, Sigma, St. Louis, Mo.) coated glass coverslips in 12-well plates for 24 hours before transfection with human BEHAB cDNA. Unfixed, unpermeabilized cultures were repeatedly rinsed in DMEM and incubated with the rabbit polyclonal anti-BEHAB antibody B6 at 4° C. for 30 minutes before fixation. Cells were subsequently fixed for 30 minutes in 4% paraformaldehyde in phosphate buffer, pH 7.4, incubated for 60 minutes with Alexa-conjugated anti-rabbit IgG secondary antibodies (Molecular Probes, Eugene, Oreg.), briefly counter-stained with DAPI (0.25 μg/ml, Sigma, St. Louis, Mo.) and prepared for fluorescence microscopy. To determine which isoform(s) of BEHAB had been detected in the cell surface by the live-cell staining procedure, transfected U87-MG cells in some wells of the same plates were rinsed in DMEM, incubated with control medium at 4° C. for 30 minutes and scraped from the wells just before the fixation step. These cells were homogeneized in 25 mM phosphate buffer, pH 7.4, and the total homogenates were prepared for protein electrophoresis.

### Glycosidase Treatments:

Soluble and particulate fractions from control brain and glioma samples were equilibrated in deglycosylation buffer (20 mM Tris HCl, 20 mM sodium acetate, 25 mM NaCl, pH 7.0) at a protein concentration of ˜1 mg/ml, and treated with the following glycosidases alone or in combination: 0.25 U/ml chondroitinase ABC, 20 mU/ml O-glycosidase from Diplococcus pneumoniae (EC 3.2.1.97, Roche, Nutley, N.J.), 100 mU/ml sialidase from Arthrobacter ureafaciens (EC 3.2.1.18, Roche, Nutely, N.J.) and 100 U/ml glycopeptidase F (PNGase F) from Chryseobacterium meningosepticum (EC 3.5.1.52, Calbiochem, La Jolla, Calif.). Sialidase was always required for efficient removal of O-linked oligosaccharides by O-glycosidase, while PNGase F alone was sufficient to remove N-linked carbohydrates. In all cases samples were incubated with the enzymes for 8 hours at 37° C. in the presence of protease inhibitors. Enzyme digestions were 8 stopped by boiling the samples in 1× gel-loading buffer.

### Western Blot Analysis:

Samples (10-15 μg total protein) were electrophoresed on reducing 6% SDS-polyacrylamide gels and proteins were electrophoretically transferred to nitrocellulose. Blots were incubated with an affinity-purified rabbit polyclonal antibody (B6) produced against a synthetic peptide corresponding to the chondroitin sulfate attachment region (amino acids 506-529) of rat BEHAB. Alternatively, BEHAB was detected with affinity-purified rabbit polyclonal antibodies produced against synthetic peptides corresponding to the amino acids 60-73 of rat BEHAB (antibody B5) and the amino acids 859-879 of human BEHAB (antibody BCRP). The antibodies B6, B5 and BCRP were previously described for the specific detection or BEHAB in rat brain samples (Matthews et al, 2000, J. Biol. Chem. 275: 22695-22703; Viapiano et al., 2003, J. Biol. Chem. 278: 33239-3347). Alkaline-phosphatase conjugated secondary antibodies were employed and the immunoreactive bands were visualized with nitro-blue tetrazolium and 5-bromo-4-chloro-3-indoyl phosphate. For densitometric quantification of BEHAB in total homogenates, immunoreactive bands were visualized by chemiluminiscence (Amersham, Piscataway, N.J.) and quantified using the Gel-Pro v3.1 software (Media Cybernetics, Silver Spring, Md.). Statistical comparisons were performed by Student's t-test with Welch's correction for non-homocedacy.

The results of the experiments presented in this Example are now described.

### Identification of Novel Isoforms of Human BEHAB Specifically Expressed in Glioblastoma:

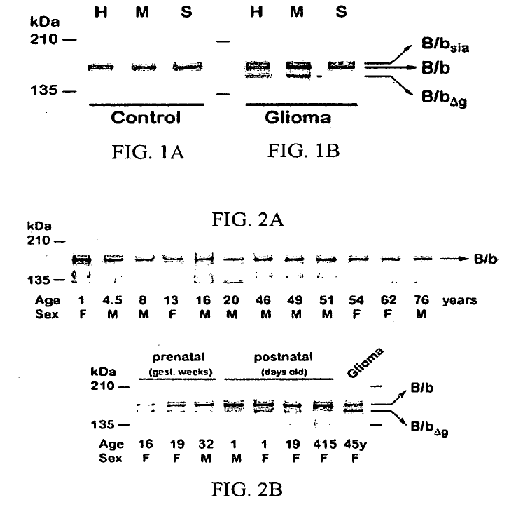

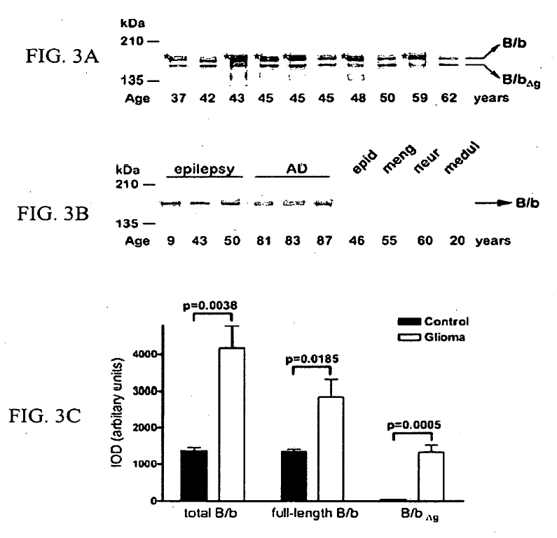

To examine the expression of BEHAB protein in normal brain and glioma, tissue was analyzed by Western blot after subcellular fractionation and enzymatic removal of chondroitin sulfate chains. In both normal brain tissue and glioma the major full-length secreted form of BEHAB ran at the expected apparent molecular mass of ˜160-kDa and was distributed in both the soluble and particulate fractions (FIG. 1). Since previous work had shown that BEHAB mRNA expression is dramatically upregulated in glioma (Jaworski et al., 1996, Cancer Res. 56: 2293-2298; Gary et al., 2000, Gene, 256: 139-147), a proportional increase in the expression of the 160-kDa BEHAB band in surgical samples from human gliomas was expected (FIG. 3C). However, surprisingly, protein analysis disclosed a more complex picture, revealing not only the increased expression of BEHAB protein but also a specific increase in the expression of specific protein isoforms in glioma. First, in approximately 50% of all the glioblastomas analyzed, the expression of protein isoforms migrating at higher apparent molecular masses than the full-length 160-kDa BEHAB were observed. This higher molecular mass form, poly-sialyated full-length BEHAB (B/bₛᵢₐ), was generated by a higher content of sialic acid on O-linked sugars in BEHAB, since both poly-sialyated full-length BEHAB and full-length BEHAB collapsed to a single position by SDS-PAGE after treatment with sialidase but not with PNGase F. Increased sialylation of proteins and glycolipids is a known malignancy-associated modification previously described in several tumors (Kim and Varki, 1997, Glycoconj J., 14: 569-576; Hakomori, 2001, Adv. Exp Med. Biol. 491: 369-402) including malignant gliomas (see for example Yamamoto et al., 1997, Brain Res., 755: 175-179; Sottocornola et al., 1998-1999, Invasion Metastasis, 18: 142-154).

Another more striking difference in the expression of BEHAB isoforms was observed in glioma. Specifically, the expression of a unique, lower molecular mass, isoform of BEHAB, denominated B/b_{Δg} (human glycosylation-variant BEHAB) was discovered (FIG. 1A). Human glycosylation-variant BEHAB migrated at an apparent molecular mass of ˜150-kDa, approximately ˜10 kDa smaller than full-length BEHAB. Unlike all other BEHAB isoforms, this isoform was found exclusively associated with membrane-containing fractions, being completely absent in the soluble fraction.

Analysis of control samples (n=14) showed that glycosylation-variant BEHAB was not detected in normal human brain at any developmental stage analyzed from 1 year of age to adulthood (FIG. 2A). A faint band migrating at a position identical to glycosylation-variant BEHAB was only observed in samples (n=6) from earlier developmental stages, from 16 weeks of gestation to 19-day-old infants (FIG. 2B). This band was barely visible in total homogenates from cortical tissue but was clearly distinguishable in the membrane-enriched fraction and was confirmed to correspond to glycosylation-variant BEHAB by enzymatic deglycosylation tests.

In marked contrast, glycosylation-variant BEHAB was present in the particulate fraction of every sample of high-grade glioma, grades III (n=2) and IV (n=19), assayed to date, without exception (FIG. 3A). To determine if glycosylation-variant BEHAB expression was unique to gliomas or also found in other neuropathologies, samples from non-glial intracranial tumors, epilepsy foci and Alzheimer's disease (n=10) were analyzed. Glycosylation-variant BEHAB was not detected in any of these samples. Overall these results indicate that, after early postnatal development, glycosylation-variant BEHAB is found uniquely in gliomas.

To compare the expression levels of the different BEHAB isoforms in normal brain and glioma, Western blots from surgical samples of glioma (n=8) and age-matched controls (n=5) were quantified by optical densitometry (FIG. 3C). Total BEHAB expression, calculated as the sum of optical densities of all full-length isoforms, was over 3-fold higher in gliomas compared to normal brain tissue. The expression of glycosylation-variant BEHAB alone accounted for roughly one-third of the total overexpression of BEHAB in glioma. These results demonstrate that a substantial proportion of all BEHAB synthesized in glioma is shunted to the pathway that makes the glycosylation-variant BEHAB isoform, resulting in a dramatic and unparalleled upregulation for this isoform since it is not observed at all in normal adult brain.

### Glycosylation-Variant BEHAB is a Full-Length Isoform of Human BEHAB

In the rodent brain several isoforms of BEHAB have been described, including isoforms with or without chondroitin sulfate chains (Yamada et al., 1994, J. Biol. Chem. 269: 10119-10126), a GPI-linked splice variant (Seidenbecher et al., 1995, J. Biol. Chem. 270: 27206-27212) and fragments generated by specific proteolytic processing (Yamada et al., 1995, Biochem. Biophys Res Commun., 216: 957-963). Thus, the observed glycosylation-variant BEHAB isoform of BEHAB could be generated by a number of different mechanisms, including alternative splicing of the mRNA and/or post-translational modifications, such as cleavage or differential glycosylation.

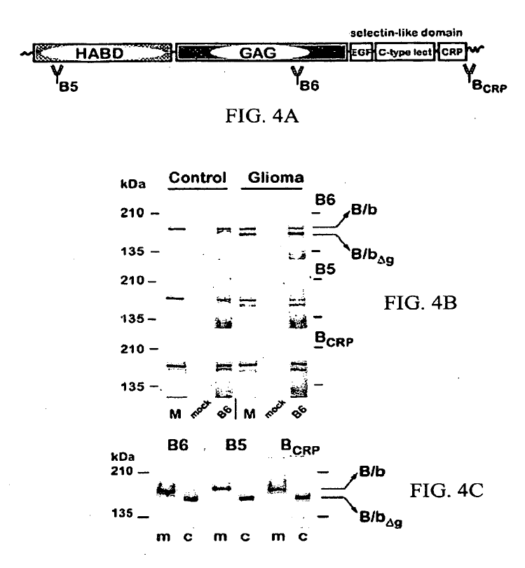

To investigate first whether the glycosylation-variant BEHAB isoform was a terminally cleaved product of full-length BEHAB or alternatively the GPI-linked splice variant two antibodies directed against epitopes located in either termini of the full-length protein were employed. The antibodies B5 and B_{CRP} detect epitopes located at less than 5 kDa from the N- and C-termini of BEHAB, respectively (FIG. 4A), and were previously characterized in the rat (Matthews et al., 2000, J. Biol Chem. 275: 22695-22703; Viapiano et al., 2003, J. Biol. Chem. 278: 33239-33247). If glycosylation-variant BEHAB was a terminally-clipped product of full-length BEHAB it would fail to be detected by at least one of these antibodies. Furthermore, the GPI-linked variant of BEHAB lacks the C-terminal CRP motif detected by B_{CRP}, thus the absence of this motif in the glycosylation-variant BEHAB isoform could alternatively indicate that it was generated by alternative splicing. To insure the specificity of the analysis, BEHAB was first immunoprecipitated from detergent extracts of human brain and human glioma membranes using a BEHAB-specific antibody, B6, prior to detection with the terminal antibodies B5 and B_{CRP}. Proteins immunoprecipitated from both normal tissue and glioma were probed with the immunoprecipitating antibody B6 as well as with B5 and B_{CRP}. All three antibodies recognized both 160-kDa full-length BEHAB as well as glycosylation-variant BEHAB from glioma samples, indicating that glycosylation-variant BEHAB is neither a terminally cleaved product of full-length BEHAB nor the GPI-linked splice variant (FIG. 4B). In addition glycosylation-variant BEHAB was never detected in immunoprecipitates from control samples, which are highly enriched in BEHAB, demonstrating that glycosylation-variant BEHAB is either not expressed at all or is expressed at extremely low levels in normal adult brain.

To investigate if glycosylation-variant BEHAB was generated from the mRNA transcript encoding the full-length isoform of BEHAB, the human glioma cell line U87-MG was transfected with a cDNA encoding secreted full-length human BEHAB and the resultant expressed proteins were analyzed by Western blotting. Transfected cells produced a 160-kDa secreted protein that was electrophoretically and immunochemically indistiguishable from the human 160-kDa full-length BEHAB isoform (FIG. 4C). In addition the transfected U87-MG cells also produced an isoform indistinguishable from glycosylation-variant BEHAB that, as in human glioma samples, localized exclusively to the particulate subcellular fraction. Again, both the glycosylation-variant BEHAB isoform and full-length BEHAB isoform were detected with all anti-BEHAB antibodies.

Together these results indicate that the glycosylation-variant BEHAB isoform was not a cleavage product of full-length BEHAB and was not created by alternative splicing of BEHAB mRNA but must, in turn, be generated by an alternative post-translational mechanism.

### Glycosylation-Variant BEHAB is Generated by Altered Glycosylation of BEHAB

Since neither alternative splicing nor cleavage were demonstrated as the mechanism responsible for generating glycosylation-variant BEHAB, other mechanisms, such as differential glycosylation or another post-translational mechanism, were investigated to determine what could account for the size difference between glycosylation-variant BEHAB and the full-length BEHAB form.

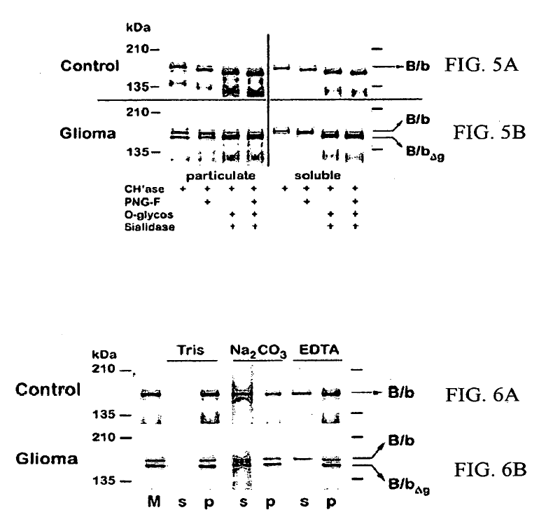

BEHAB carries N-linked and O-linked oligosaccharides. In addition, BEHAB is known as a part-time proteoglycan because there are isoforms that exist both with and without chondroitin sulfate chains. Treatment with chondroitinase ABC, which removes chondroitin sulfate chains from BEHAB, enhanced the immunoreactivity of full-length BEHAB both in soluble and particulate fractions from controls and glioma samples, which was an expected result since such treatment causes the forms of the protein carrying chondroitin sulfate chains to collapse into a single band. However, surprisingly, this treatment did not affect the apparent molecular mass or immunoreactivity of glycosylation-variant BEHAB. Furthermore, treatment of the same fractions with a combination of chondroitinase and enzymes that remove N- and O-linked sugars shifted the full-length BEHAB band towards the position of glycosylation-variant BEHAB both in control and glioma samples (FIG. 5). In contrast, the electrophoretic mobility of glycosylation-variant BEHAB in the particulate fraction of gliomas was not affected by the treatment with glycosidases, indicating that this isoform lacked the N- and O-linked sugars present in the glycosylated, full-length form. These results provide evidence that the size difference between the 160-kDa isoform of BEHAB and the glycosylation-variant isoform found in glioma are likely due only to differences in glycosylation.

Glycosylation-Variant BEHAB is Associated with Human Glioma Membranes by a Unique Mechanism

As described elsewhere herein, and depicted in FIGS. 1 through 4, the underglycosylated form of BEHAB, glycosylation-variant BEHAB, partitions exclusively with the particulate fraction of glioma. The mechanism of the association between the membrane and glycosylation-variant BEHAB was therefore explored.

To determine whether glycosylation-variant BEHAB could be associated with the cell surface through a lipid-linkage or as an integral membrane protein, membranes from normal brains and gliomas were treated with sodium carbonate, which releases peripherally associated but not GPI-linked or integral membrane proteins. As depicted in FIG. 6, both the glycosylated full-length BEHAB and the underglycosylated BEHAB forms of BEHAB were released into the soluble fraction by sodium carbonate treatment in similar proportions, indicating that both forms are peripherally-associated proteins.

The only previously described mechanism by which BEHAB associates with the cell surface is a calcium-dependent binding through its lectin-like domain (Aspberg et al., 1997, Proc Natl Acad Sci USA 94: 10116-10121; Miura et al., 1999, J. Biol. Chem. 274: 11431-11438). Accordingly, membranes from normal brains and gliomas were treated with EDTA in order to disrupt calcium-dependent binding. EDTA treatment partially released the 160-kDa full-length isoform of BEHAB both in normal tissue and in glioma (FIG. 6). In contrast, the glycosylation-variant BEHAB isoform in glioma was not released at all by EDTA, demonstrating that glycosylation-variant BEHAB is associated to the cell membranes by a calcium-independent mechanism.

### Glycosylation-Variant BEHAB is Expressed on the Cell Surface

One possible explanation for the biochemical partitioning of glycosylation-variant BEHAB with the membrane fraction would be that it represents a mis-folded, incompletely glycosylated form, which is retained in the secretory pathway and does not reach the cell surface.

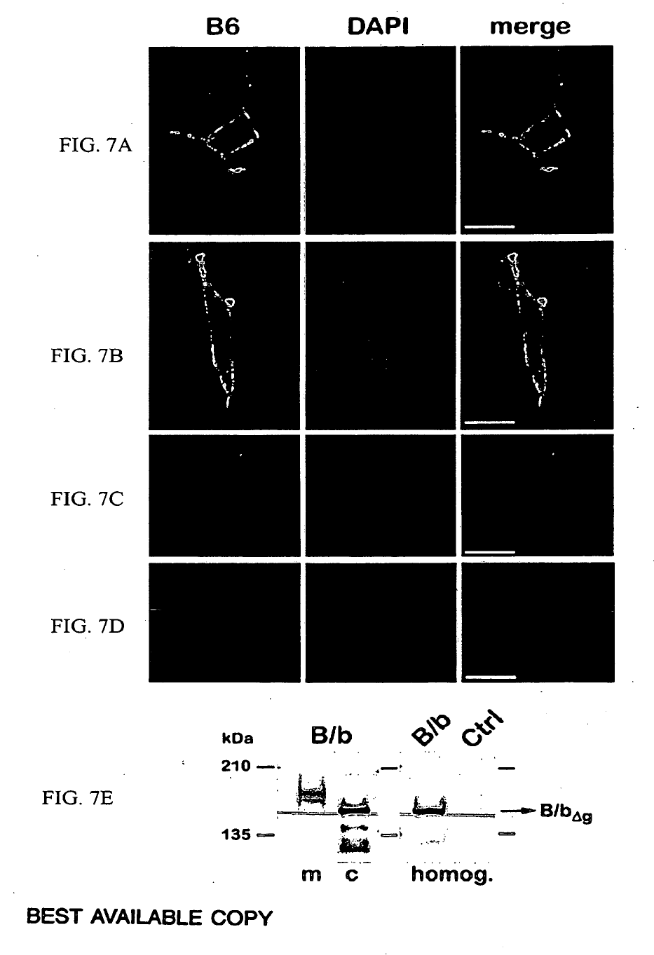

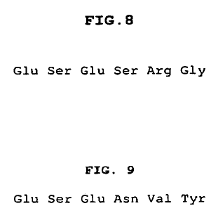

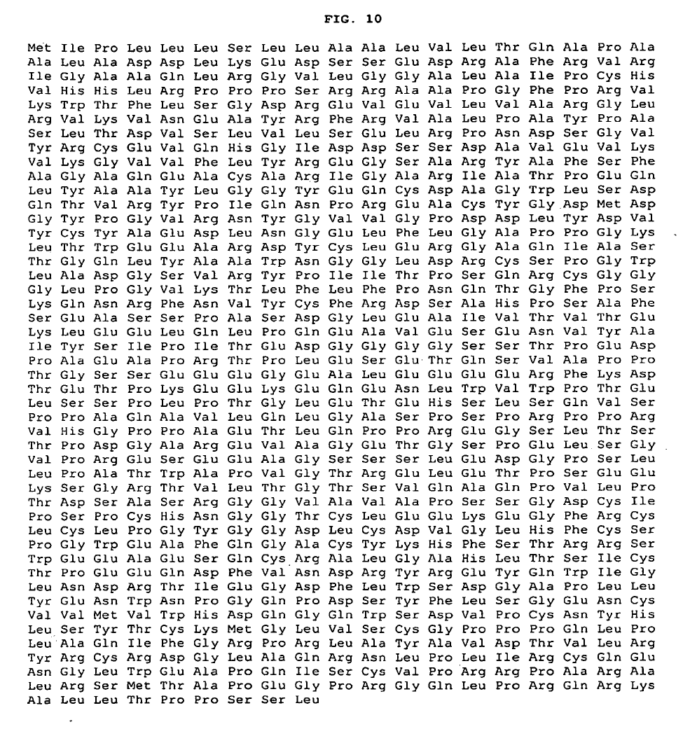

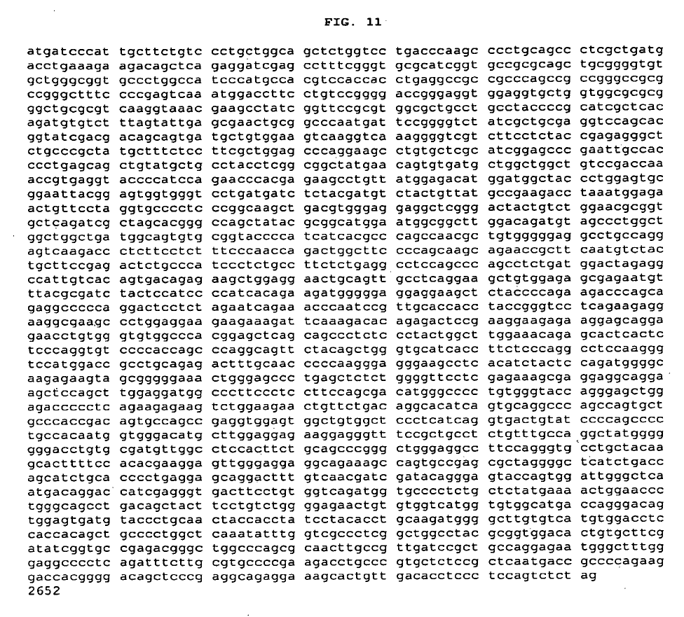

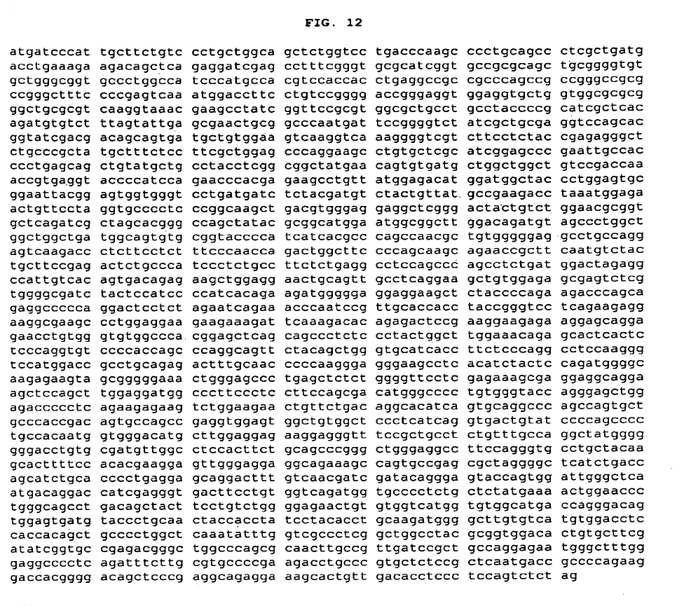

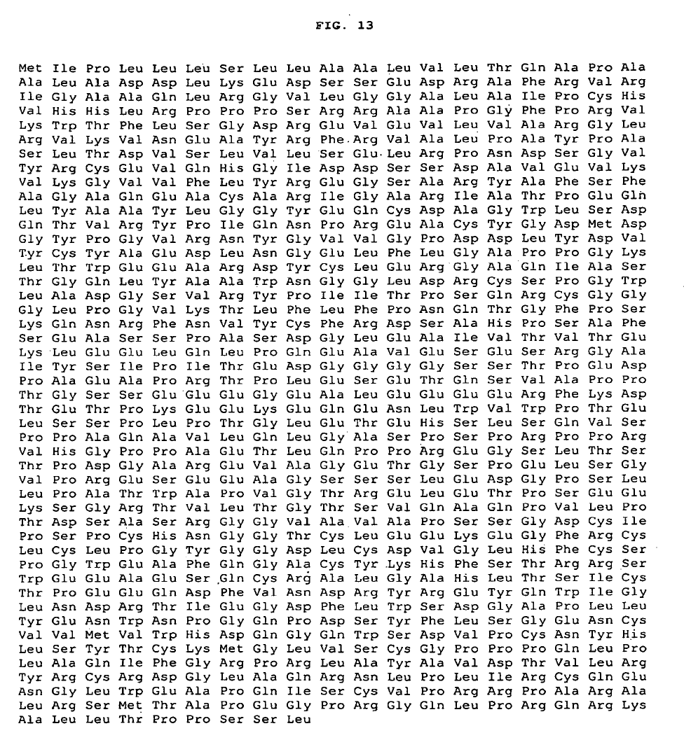

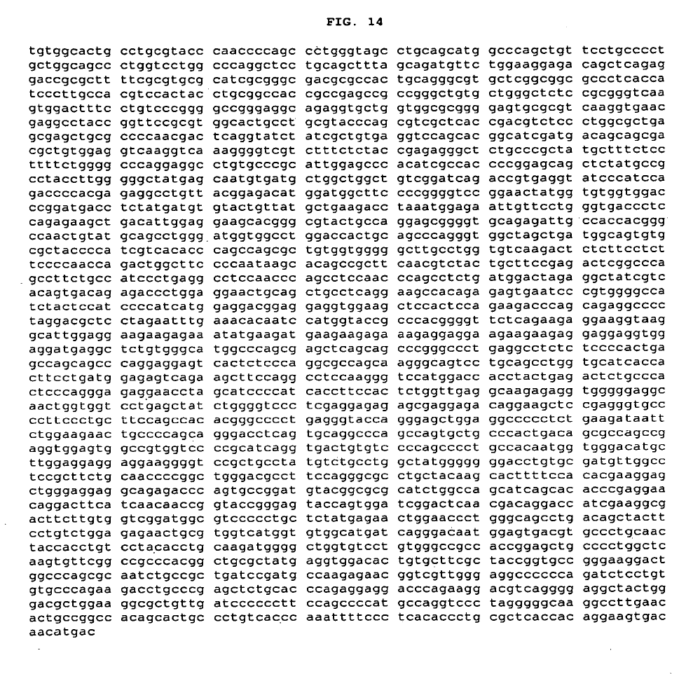

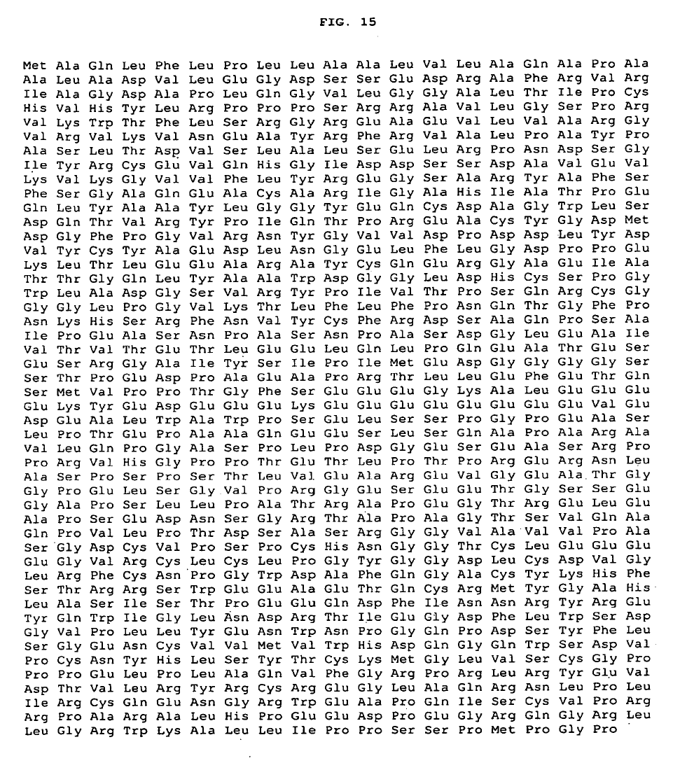

To determine whether glycosylation-variant BEHAB is localized on the extracellular surface of cells, U87-MG cells were transfected with cDNA for full-length human BEHAB and subsequently rinsed and stained with an anti-BEHAB antibody before fixation. Immunodetection using this technique allows only epitopes on the extracellular surface of the cell to be recognized, since the antibody is excluded from the interior of the cell. These studies revealed BEHAB staining on the extracellular surface of transfected cells (FIG. 7A-D). However, since U87-MG cells express both 160-kDa BEHAB and glycosylation-variant BEHAB, the isoform actually present on the cell surface was examined.

Cells were processed identically as the stained cells but collected without fixation in order to determine the identity of the isoform(s) detected by live-cell staining. Western blotting of total homogenates showed that only glycosylation-variant BEHAB remained in these cells after the initial rinsing for live-cell staining procedures. Thus, glycosylation-variant BEHAB is the form detected in the cell surface while 160-kDa BEHAB is secreted and not associated at all with the cell surface in cultured cells (FIG. 4C).

### Discussion

### Glycosylation-Variant BEHAB is a Novel, Glioma-Specific Isoform of BEHAB in the Adult Brain

Disclosed herein is the identification of a novel underglycosylated isoform of BEHAB that is restricted to high-grade glioma and is also the major upregulated isoform of BEHAB in high-grade glioma.

Previous studies had demonstrated that BEHAB mRNA levels are dramatically upregulated in human glioma and in a rat experimental model of glioma (Jaworski et al., 1996, Cancer Res. 56: 2293-2298; Gary et al., 2000, Gene, 256: 139-147). The results disclosed herein indicate that this upregulation leads not only to a general upregulation of the expression of BEHAB but also to the specific upregulation of unique isoforms of BEHAB in glioma. These isoforms (poly-sialyated full-length BEHAB, glycosylation-variant BEHAB) are characterized by differential glycosylation, the most consistent and obvious being the membrane-associated glycosylation-variant BEHAB isoform (FIGS. 1-3). This novel isoform seems to lack all detectable carbohydrate additions to the core protein (FIG. 5).

Glycosylation-variant BEHAB was detected in all samples of high-grade glioma that were analyzed to date. Interestingly, glycosylation-variant BEHAB is absent in other neuropathologies such as Alzheimer's disease, epileptic foci and several non-glial intracranial tumors (FIG. 3B). Thus the appearance of this isoform does not likely represent a general pathogenic or gliotic process but instead represents a specific modification in high-grade gliomas.

In stark contrast with gliomas, the glycosylation-variant BEHAB isoform was never detected in normal adult human brain. Glycosylation-variant BEHAB is weakly expressed only during prenatal and early postnatal development, but disappears by the first year of age and is completely absent from tissue thereafter. These data suggest that expression of glycosylation-variant BEHAB in gliomas could represent a recapitulation of early developmental programs, a mechanism that has previously been implicated in glioma progression Seyfried, 2001, Perspect. Biol Med., 44: 263-282). However, the expression of glycosylation-variant BEHAB in gliomas is at a much higher level than has been observed at any stage of human CNS development.

Glycosylation-variant BEHAB is thus a novel specific marker of high-grade glioma in adult human brain. This is a significant finding since there are very few proteins or protein isoforms whose expression is specifically restricted to glioma (Kurpad, et al., 1995, Glia, 15: 244-256). While the overexpression of many proteins has been described in glioma, the expression of specific proteins or protein isoforms in tumors is relatively rare. The specific expression of glycosylation-variant BEHAB in gliomas and its cell-surface localization make it an interesting potential target for glioma immunotherapy.

### Glycosylation-Variant BEHAB is a Full-Length Isoform of BEHAB

Presented herein is clear evidence that glycosylation-variant BEHAB is, unexpectedly, a full-length product of BEHAB mRNA, like the 160-kDa isoform that appears in normal human brain.

Glycosylation-variant BEHAB is not a cleavage product of BEHAB since, although is 10-12 kDa smaller than the 160-kDa form it is nevertheless recognized by antibodies that detect epitopes located at less than 5 kDa from the N- and C-termini of full-length BEHAB (FIG. 4B). Similarly, glycosylation-variant BEHAB is not a splice variant of full-length BEHAB since it is expressed from the full-length human BEHAB cDNA as observed in transfected U87-MG cells in culture (FIG. 4C). These two independent demonstrations also rule out any possibility that glycosylation-variant BEHAB could be the GPI-linked isoform of BEHAB previously reported in rat and human brain. In addition, glycosylation-variant BEHAB is released from the glioma membranes with sodium carbonate (FIG. 6), a treatment that removes only peripherally bound proteins but not those attached by GPI-anchors.

The human GPI-linked BEHAB protein isoform was not detected in the data disclosed herein. In the rat brain this isoform, in the presence of its GPI-anchor, migrates in SDS-PAGE at the same position of the rat full-length, glycosylated BEHAB isoform and is therefore masked by this major isoform (Viapiano et al., 2003, J. Biol. Chem., 278: 33239-33247). However in human tissue the lack of detection seems to be more likely due to the very low level of expression of human GPI-BEHAB mRNA compared to the full-length isoform (Gary et al., 2000, Gene, 256: 139-147).

### Glycosylation-Variant BEHAB has Unique Properties Among the Known BEHAB Isoforms

The most important characteristic of glycosylation-variant BEHAB is that it appears to completely lack attached carbohydrates (FIG. 5). Despite this, glycosylation-variant BEHAB is localized to the extracellular surface (FIG. 7), demonstrating that it is not just a core-protein precursor sequestered inside the cell. In addition, the association of glycosylation-variant BEHAB with the plasma membrane seems to be mediated by a calcium-independent mechanism (FIG. 6), distinct from those previously described for other BEHAB isoforms. This association may likely involve ligands different from the ones known for full-length glycosylated BEHAB.

Interestingly, a BEHAB isoform in the rat brain, named B/b₁₃₀ was recently described (Viapiano et al., 2003, J. Biol. Chem., 278: 33239-33247), with essentially identical biochemical characteristics as glycosylation-variant BEHAB. Although, in contrast to glycosylation-variant BEHAB, B/b₁₃₀ is expressed as a minor form in normal adult rat brain, it is also the major upregulated form of BEHAB in rat experimental gliomas. This result indicates that the upregulation of this underglycosylated variant of BEHAB is common in rat and human glioma, therefore suggesting that the glycosylation state of BEHAB may play a significant role in the progression of glial tumors.

### Role of BEHAB Neoglycoforms in Glioma

Aberrant glycosylation of cell surface proteins occurs in almost all cancers (Hakomori, 2002, Proc. Natl. Acad Sci USA 99: 10231-10233). Changes in glycosylation disrupt the normal protein-protein interactions and therefore can be associated to tumor invasion and metastasis (Kim and Varki, 1997, Glycoconj. J. 14: 569-576; Gorelik et al., 2001, Cancer Metastasis Rev. 20: 245-277).

Aberrant glycosylation is identified by the appearance of either truncated versions of normal oligosaccharides or unusual types of terminal oligosaccharide sequences (e.g., Lewis^{x/a}). These changes may equally affect N- and O-linked oligosaccharides (Burchell et al., 2001, J. Mammary Gland Biol Neoplasia 6: 355-364 2001; Dwek et al., 2001, Proteomics 1: 756-62). In particular, a general increase in the appearance of alpha 2,6- and alpha 2,3-linked sialic acid is a common feature of tumors (Narayanan, 1994, Ann Clin Lab Sci. 24: 376-384), including glioma (Reboul et al., 1996, Glycoconj. J. 13: 69-79; Yamamoto et al., 1997, Brain Res., 755: 175-179), and has been associated to an increase in the metastatic ability of certain cancers. In agreement with these observations, disclosed herein is a sialylated neoglycoform of BEHAB, poly-sialyated full-length BEHAB, in roughly half of all our samples of high-grade glioma, in addition to the isoform glycosylation-variant BEHAB (FIG. 3A). BEHAB may be an important target of altered sialylation in glioma as has been described for other cell-surface glycoproteins. This implies that the appearance of poly-sialyated full-length BEHAB could be associated to clinical outcome.

Lack of specific oligosaccharides in tumors, though less commonly noted, has also been described (see Dennis, 1986, Cancer Res. 46: 4594-4600; Dabelsteen et al., 1991, J. Oral Pathol Med. 20: 361-368; Ciborowski and Finn, 2002, Clin. Exp Metastasis 19: 339-45). An interesting example is represented by the cell-surface receptors with aberrantly underglycosylated neo-glycoforms in CNS tumors that cannot bind their normal ligands (e.g., CD44H in neuroblastoma (Gross et al., 2001, Med. Pediatr Oncol. 36: 139-41). In a similar manner, it is possible that the overexpression of the underglycosylated isoform glycosylation-variant BEHAB on the surface of glioma cells could promote tumor progression by disturbing the interactions of normal BEHAB and enabling novel cell-cell interactions that favor invasion.

BEHAB is invested with a diverse set of carbohydrates that can be modified in glioma. Changes in the expression of specific glycosyltransferases and/or modification of metabolic pathways could result in multiple specific carbohydrate modifications, thus generating novel glycoforms of BEHAB, of which the poly-sialyated full-length BEHAB isoform is a clear example. However, it is more difficult to understand the mechanism by which the glycosylation-variant BEHAB isoform is made, since it seems to have essentially a complete absence of attached sugar chains. BEHAB is typically invested with N-linked sugars, mucin-type O-linked sugars and often chondroitin sulfate chains. Despite the fact that these different carbohydrates are added by different arrays of enzymes, they all appear to be absent from the glycosylation-variant BEHAB isoform. This suggests that the glycosylation-variant BEHAB isoform is generated by a unique mechanism that globally effects the ability of any carbohydrates to be added to the protein core, yet it is a mechanism that specifically targets BEHAB (and perhaps other proteins) while the glycosylation of most proteins remains relatively normal.

Independently of how this unique BEHAB glycoform is generated, it is important to note that the lack of glycosylation imparts unique binding and functional characteristics to the protein core. Accordingly, glycosylation-variant BEHAB could play a unique functional role in glioma. Understanding how glycosylation-variant BEHAB arises and associates with the cell membrane will provide insight on its possible functional roles in glioma development, and increase our understanding of these brain tumors, opening novel therapeutic avenues to the treatment of glioma.

Targeting tumor cells selectively through their specific cell-surface antigens is an approach that is regaining popularity as a cancer therapy. Considerable research over the past decade has made great progress in demonstrating the utility of antibody immunotherapy in the treatment of many tumor types (see Carter, 2001, Nat. Rev Cancer, 1: 118-129), including glioma (Kurpad et al., 1995, Glia 15: 244-256; Kuan et al., 2001, Endocr. Relat Cancer, 8: 83-96; Goetz et al., 2003, J. Neurooncol. 62: 321-328). Given the refractary properties of gliomas to traditional chemo- and radiotherapy, immunotherapy is a promising treatment for primary CNS tumors. However, a hurdle in using this approach as a therapy for glioma has been the lack of good cellular targets that are both restricted to the tumor cells and available at the cell surface for targeting (Yang et al., 2003, Cancer Control. 10: 138-147). Among those that have been proposed (Kurpad et al., 1995, Glia 15: 244-256), and clinically explored are the deletion mutant EGF receptor (EGFRvIII, reviewed in Kuan et al., (2001, Endocr. Relat Cancer, 8: 83-96)), which is expressed in ˜50% of all glioblastomas (Kurpad et al., 1995, Glia 15: 244-256), and the extracellular matrix protein tenascin-C, which is highly upregulated in >90% of all gliomas compared to normal brain (McLendon et al., 2000, J. Histochem Cytochem. 48: 1103-1110).

In this respect, BEHAB is an interesting target for therapy since its upregulation and further protein cleavage play an enhancing role in experimentally induced tumor progression (Zhang et al., 1998, J. Neurosci. 18: 2370-2376; Nutt et al., 2001, Cancer Res. 61: 7056-7059). Indeed, BEHAB has previously been proposed as a potential tumor target (Nutt et al., 2001, Neuroscientist 7: 113-122). However, its presence in the normal adult human brain and the fact that full-length glycosylated BEHAB is a secreted ECM molecule create concerns of, respectively, therapeutic risk and potentially reduced effectiveness that would complicate its molecular targeting. The identification of the novel glycosylation-variant BEHAB isoform resolves these theoretical considerations and presents a novel and specific molecular target. As disclosed herein, glycosylation-variant BEHAB is fully restricted to glioma cells and it is not detected in the normal adult human nervous tissue. In addition, the strict localization of glycosylation-variant BEHAB to the cell surface suggests that it may be an ideal target for glioma immunotherapy.

In sum, as demonstrated and disclosed by the data herein, a novel isoform of the CNS-specific protein BEHAB has been identified. Further, as these data show, glycosylation-variant BEHAB is restricted to glioma, represents an under- or un-glycosylated form of BEHAB and is specifically bound to the plasma membrane of the transformed glial cells. Understanding the functional role of this novel isoform and directly targeting this isoform may present novel strategies for glioma therapy.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated human poly-sialyated BEHAB polypeptide, wherein said ply-sialyated BEHAB polypeptide has a molecular weight greater than about 160 kDa and comprises the amino acid sequence set forth in SEQ ID NO:8.

2. The isolated polypeptide of claim 1, wherein said molecular weight is from about 163 kDa to about 166 kDa.

3. The isolated polypeptide of claim 1, wherein said polypeptide comprises from about 10 to about 20 sialic acid residues more that full-lenght BEHAB.

4. The isolated polypeptide pf 3, wherein said sialic acid residues are attached to said polypeptide via an O-linkage.

5. A method of detecting a malignant glioma in a mammal, said method comprising contactin a biological samle of said mammal with an antibody that specifically binds with a glycosylation-variant BEHAB polypeptide and detecting binding of said antibody to said biological sample, wherein binding of said antibody with said biological sample detects a malignant glioma in a mammal.

6. The method of claim 5, wherein said mammal is a human.

7. The method of claim 5, wherein said biological sample is a CNS tissue sample.

8. The method of claim 7, wherein said CNS tissuee sample is a brain tissue.

9. The method of claim 5, wherein said antibody is selected from the group consisting of B5, B6, and B_{CRP}.

10. The method of claim 9, wherein said said antibody comprises a tag covalently linked thereto.

11. The method of claim 5, wherein said glioma is a malignant high grade glioma.

12. A method of differentially diagnosing a malignant glioma from a benign glioma in a mammal, said method comprising contacting a biological sample of said mammal with an antibody that specifically binds with a glycosylation-variant BEHAB polypeptide and detecting binding of said antibody to said biological sample, wherein binding of said antibody with said biological sample detects a malignant glioma in a mammal.

13. The method of claim 12, wherein said mammal is a human.

14. The method of claim 12, wherein said biological sample is a CNS tissue sample.

15. The method of claim 14, wherein said cns tissue sample is a brain tissue.

16. The method of claim 12, wherein said antibdody is selected from the group consisting of B5, B6, and B_{CRp}.

17. The method of claim 16, wherein said antibody comprises a tag covalently linked therto.

18. The method of claim 12, wherein said malignant glioma is a malignant high grade glioma.

19. The method of claim 12, wherein said benign glioma is a benign low grade glioma.

20. The method of claim 19, wherein said benign low grade glioma is a grade II glioma.

21. The method of claim 19, wherein said benign low grade glioma is an oligodendroglioma associated with chroniv epilepsy.

22. A method os assessing a change in tumor progression in a mammal, said method comprising contacting a first biological sample of said mammal and detecting binding of said antibody to said biological sample, said method further comprising comparing the level of glycoslation-variant BEHAB in a second biological sample sample with the level of glycosylation-variant BEHAB in a second biological sample from said mammal, wherein a difference in the level of glycosylation-variant BEHAB in said first biological sample indicaties a change in tumor progression in said mammal.

23. The method of claim 22, wherein said mammal is a human.

24. The method of claim 22, wherein said biological sample is a CNS tissue sample.

25. The method of claim 24, wherein said CNS tissue sample is a brain tissue.

26. The method of claim 22, wherein said antibidy is selcted from the group consisting of B5, B6, and B_{CRP}.

27. The method of claim 22, wherein said antibody comprises a tag covalently linked thereto.

28. The method of claim 22, wherein said glioma is a malignant high grade glioma

29. A kit for detecting a malignant glioma, said kit comprising an antibody that specifically binds with a glycosylation-variant BEHAB, said kit further comprising an applicator, and an instructional material for use thereof.

30. A kit for differentially diagnosing a malignant glioma from a benign glioma, said kit comprising an antibody that specifically binds with a glycosylation-variant BEHAB, said kit further comprising an applicator, and an instructional material for use thereof.

31. A kit for assessing a change in tumor progresion in a mammal, said kit comprising an antibody that specifically binds with a glycosylation-variant BEHAB, said kit further comprising an applicator, and an instructional material for use thereof.
